(19) United States
(12) Patent Application Publication
(10) Pub. No.: US 2007/0150063 A1
(43) Pub. Date: Jun. 28, 2007

(54) Devices for intervertebral augmentation and methods of controlling their delivery

(75) Inventors: Ramon A. Ruberte, Ann Arbor, MI (US); Michael J. O'Neil, West Barnstable, MA (US)

Correspondence Address: NUTTER MCCLENNEN & FISH LLP, WORLD TRADE CENTER WEST, 155 SEAPORT BOULEVARD, BOSTON, MA, 02210-2604, US

(73) Assignee: DePuy Spine, Inc., Raynham, MA (US)

(21) Appl. No.: 11/316,343

(22) Filed: Dec. 22, 2005

(51) Int. Cl. A61F 2/44

(52) U.S. Cl. 623/17.16; 623/23.76

(57) ABSTRACT

Devices and methods for treating diseased or damaged portions of an intervertebral region are provided. In particular, intervertebral implants that can include use of a tissue regeneration structure having small intestine submucosa are described. The intervertebral implants can be utilized with any combination of load bearing structures for supporting loading on the implant, shaping structures for biasing the configuration of the implant, collapsible support structures for shaping the implant, and other features. Implants can also be formed with an enclosure to contain a filling material, such as an injectable small intestine submucosa formulation. Methods of delivering and utilizing the various implants are also discussed.

### RELATED APPLICATIONS

This application is related to copending U.S. patent application Ser. No. ______ filed Dec. 22, 2005 entitled “Devices for Intervertebral Augmentation” having inventors Ramon A. Ruberte, Michael J. O'Neil, and Patrick G. DeDeyne, and copending U.S. patent application Ser. No. ______ filed Dec. 22, 2005 entitled “Methods and Devices for Intervertebral Augmentation Using Injectable Formulations and Enclosures” having inventors Ramon A. Ruberte and Michael J. O'Neil, the contents of which are both hereby incorporated herein by reference in their entirety.

### FIELD OF THE INVENTION

The present invention is directed broadly to methods and devices for treating back pain and other ailments caused by defects or disease to portions of an intervertebral region.

### BACKGROUND OF THE INVENTION

Injury and/or degeneration of the intervertebral disc can cause back pain as a result of disc herniation, rupture of the annulus and/or prolapse of the nucleus pulposus. Herniation and nucleus prolapse can cause spinal canal and foraminal stenosis. All may cause release of chemotactic factors that irritate the spinal cord. Acute damage to the annulus and/or nucleus prolapse can cause abnormal biomechanical function of the disc and subsequent disc degeneration.

Discectomy, laminectomy, laminotomy and/or spine fusion procedures represent state of the art surgical treatment for disc problems. Heating the disc using a probe has been suggested to “weld” defects. Injecting curable materials into the nucleus has also been suggested to act as filler material for the nucleus and annular defect. As well, a number of prosthetic devices have also been introduced that can act as a replacement for an intervertebral disc, or a portion thereof.

Despite the presence of these and other treatments, a need persists for improved devices that aid in the treatment intervertebral damage or disease. As well, associated methods of treatment, as well as procedures for delivering the aforementioned devices, preferably by minimally invasive techniques, can also contribute to improved care of the intervertebral region.

### SUMMARY OF THE INVENTION

The present invention generally provides methods and devices for treating damaged or diseased intervertebral regions. Some embodiments of the invention are drawn to intervertebral implants that include a tissue regeneration structure and a load bearing structure coupled thereto. The tissue regeneration structure can promote tissue growth and can include at least one layer of small intestine submucosa. The load bearing structure can support a load on the implant and includes a load bearing material with a higher compressive modulus than small intestine submucosa subsequent to implantation in a patient. Resorbable and/or non-resorbable materials can be utilized as a load bearing material. Alternatively, a shape retaining structure can be coupled to the tissue regeneration structure for support loading on the implant. The shape retaining structure can be more resistant to shape change under loading that the tissue regeneration structure after the implant is positioned within a patient.

Implants can be adapted to replace portions or an entirety of an intervertebral disc, and/or at least partially seal an opening in an intervertebral structure. Implants can include an ingrowth promoting material that contacts at least one of the tissue regeneration structure and the load bearing structure. Suitable ingrowth promoting materials can include one or more of biofactors, cells, and an osteoinductive material for promoting bony tissue ingrowth and implant attachment. Implants can alternatively or additionally include a tissue contacting material can be an anti-adhesive material or an adhesive material. Some implants can include at least one securing device that may include small intestine submucosa. The securing device is effective to attach the intervertebral implant to tissue (e.g., bone, the annulus fibrosis). The implant can further include one or more tab structures that may include small intestine submucosa. A tab structure can be adapted to extend from a portion of the intervertebral implant and be effective to receive a securing device for attaching the intervertebral implant to tissue.

In another embodiment, the load bearing structure of an intervertebral implant includes one or more layers of load bearing material, which can be adapted, along with one or more layers of a tissue regeneration structure, to form a laminate structure or a portion thereof. Laminate structures can be prefabricated or folded into an implantable structure that is adapted to be implanted within an intervertebral space. A laminate structure can also be adapted to form a coiled configuration. Laminate structures may also include a material for promoting tissue ingrowth. A laminate structure can also be configured as a set of nested bands.

One or more additional layers can be added to a laminate structure. For example, an end layer including small intestine submucosa can be coupled to an opposed end of the laminate structure so that the end layer is oriented substantially perpendicular to a direction of the surfaces of the laminate structure. A layer of the load bearing material in the laminate structure can be adapted to form a non continuous layer structure, which is optionally embedded in a matrix. Small intestine submucosa layers in a tissue regeneration structure can have fibers that are substantially aligned in a predetermined direction. For example, a tissue regeneration structure can include two or more layers of SIS with a first layer having fibers substantially aligned in one direction and a second layer having fibers substantially aligned is a different direction relative to the first layer.

In another embodiment, an intervertebral implant can include a tissue regeneration structure as at least a portion of a multilayered structure having adjacently located surfaces and a load bearing structure including at least one block structure embedded within the multilayered structure. The load bearing structure can further include at least one layer of load bearing material adapted into the multilayered structure. The multilayered structure can also include any combination of the features described herein for the implants utilizing a laminate structure (e.g., including one or more end layers, adapted to include a plurality of nested bands or to form a coiled configuration). In particular, when the multilayered structure forms a coiled configuration, a block structure can be positioned substantially in the center of the coiled configuration. Alternatively, or in addition, multiple block structures can be positioned within the multilayered structure.

### BRIEF DESCRIPTION OF THE DRAWINGS

The invention will be more fully understood from the following detailed description taken in conjunction with the accompanying drawings, in which:

FIG. 1A depicts a cross-sectional side view of the width of an intervertebral implant having a tissue regeneration structure and a load bearing structure embodied as a laminate structure consistent with an embodiment of the invention;

FIG. 1B presents a photograph showing the layers of materials in an intervertebral implant;

FIG. 1C presents a photograph of a sample of small intestine submucosa being formed into an exemplary layer;

FIG. 1D presents a photograph showing a perspective view of the intervertebral implant shown in FIG. 1B;

FIG. 2A depicts a perspective view of a portion of an intervertebral implant having a layer embodied as a plurality of fibers;

FIG. 2B depicts a cross sectional view of an intervertebral implant having three layers embodied as a plurality of fibers;

FIG. 3 depicts a perspective view of a portion of an intervertebral implant having layers embodied as a plurality of fibers oriented in two perpendicular directions;

FIG. 4 depicts a cross sectional view of the intervertebral implant having a layer embodied as a plurality of layers embedded in a matrix of material;

FIG. 5 depicts a perspective view of a layer of an intervertebral implant embodied as a woven mesh of fibers;

FIG. 6A depicts a cross-sectional view of an exemplary intervertebral implant having a laminate structure with a layer including osteoconductive materials;

FIG. 6B depicts a cross-sectional view of an exemplary intervertebral implant having a laminate structure with an end layer including osteoconductive materials;

FIG. 6C depicts a cross-sectional view of an exemplary intervertebral implant having a laminate structure with a layer including an anti-adhesive material;

FIG. 6D depicts a cross-sectional view of an exemplary intervertebral implant having a laminate structure with a layer including an adhesive material;

FIG. 7A depicts a cross-sectional view of an exemplary intervertebral implant having a laminate structure including sets of pairs of small intestine submucosa layers interrupted by layers of load bearing material;

FIG. 7B depicts a cross-sectional view of an exemplary intervertebral implant having a laminate structure including sets of pairs of small intestine submucosa layers interrupted by layers of load bearing material, and layers having tissue ingrowth materials;

FIG. 7C depicts a cross-sectional view of the exemplary intervertebral implant shown in FIG. 7B including an end layer acting as a sealing device for the implant;

FIG. 7D depicts a cross-sectional view of an exemplary intervertebral implant having a laminate structure including sets of pairs of small intestine submucosa layers interrupted by layers of load bearing material;

FIG. 8A depicts a transverse view of a disc space between two vertebral bodies having an intervertebral implant located therein;

FIG. 8B depicts an axial view of the disc space in an annulus fibrosis between the two vertebral bodies shown in FIG. 8A, the intervertebral implant being a laminate structure adapted in a coil configuration, the coil configuration having an axis in the direction of the axis of the vertebrae;

FIG. 8C depicts an axial view of the disc space in an annulus fibrosis between the two vertebral bodies shown in FIG. 8A, the intervertebral implant being a laminate structure adapted in a folded configuration, the folded configuration having creases aligned in the direction of the axis of the vertebrae;

FIG. 8D depicts a transverse view of a disc space between two vertebral bodies having an intervertebral implant being a laminate structure adapted in a folded configuration, the folded configuration having creases aligned perpendicular to the direction of the axis of the vertebrae;

FIG. 8E depicts a transverse view of a disc space between two vertebral bodies having an intervertebral implant being a laminate structure adapted in a coiled configuration, the coiled configuration having an axis aligned perpendicular to the direction of the axis of the vertebrae;

FIG. 8F depicts an axial view of a disc space in an annulus fibrosis between the vertebral bodies shown in either FIG. 8D or 8E filled with an intervertebral implant;

FIG. 9A depicts a side view of an intervertebral implant having a laminate structure in a folded configuration;

FIG. 9B depicts a perspective view of an intervertebral implant having a laminate structure in a coiled configuration;

FIG. 9C depicts a perspective cutaway view of a portion of an intervertebral implant having a laminate structure in a coiled configuration, the laminate structure including two layers of small intestine submucosa having fibers that are aligned in different directions;

FIG. 9D depicts a perspective view of an intervertebral implant having a laminate structure in a coiled configuration that includes an end layer;

FIG. 10A depicts a perspective view of a cylindrical structure used to form intervertebral implants having a laminate structure with layers configured in a nested band structure, the cylindrical structure capable of being cut into three separate implants;

FIG. 10B depicts a perspective view of an intervertebral implant from the cylindrical structure of FIG. 10A, the implant having bands of small intestine submucosa with different alignments of fibers;

FIG. 11 depicts a perspective view of an intervertebral implant having a load bearing structure embodied as a core with a layer structure wrapped around the core;

FIG. 12 depicts a perspective view of an intervertebral implant having a tissue regeneration structure embodied as a layer of small intestine submucosa and a load bearing structure embodied as a core and a layer of load bearing material coupled to the layer of small intestine submucosa, the layers wrapped around the core;

FIG. 13A depicts a perspective view of an intervertebral implant having a plurality of block structures embedded with a matrix wound around a core, the axes of the block structures oriented in the same direction as the axis of symmetry of the implant;

FIG. 13B depicts a perspective view of an intervertebral implant having a plurality of block structures embedded with a matrix wound around a core, the block structures oriented to coil around a core of the implant;

FIG. 14A depicts a cutaway perspective view of a cylindrical block structure that penetrates through a matrix of an intervertebral implant;

FIG. 14B depicts a cutaway perspective view of two cylindrical block structures that partially penetrates through a matrix of an intervertebral implant;

FIGS. 15A-15F depict perspective views of various exemplary shapes for block structures embedded in matrix material that can be utilized with intervertebral implants consistent with embodiments of the invention;

FIG. 16A depicts a perspective view of an intervertebral implant having a laminate structure in a coiled configuration having tab structures for securing the implant;

FIG. 16B depicts a perspective view of an intervertebral implant having a laminate structure in a flat configuration having tab structures for securing the implant;

FIG. 16C depicts a cross-sectional transverse view of an intervertebral disc space having a intervertebral implant in a folded configuration, the implant being secured by its tab structures that are adhered to a portion of an annular fibrosis;

FIG. 16D depicts a cross-sectional transverse view of an intervertebral disc space having a intervertebral implant, the implant being secured by its tab structures that are attached to vertebrae by securing devices;

FIG. 17A depicts a side view of a securing device having a layer of resorbable material incorporated on the securing device's head;

FIG. 17B depicts a ghosted, side view of a securing device having an internal volume for holding materials;

FIG. 17C depicts a side view of a securing device;

FIG. 17D depicts a perspective view of a covering device having a covering layer coupled to two securing devices;

FIG. 17E depicts a perspective view of a covering device having a covering layer coupled to four securing devices, the covering layer constructed from weaved strips of resorbable and non-resorbable materials;

FIG. 17F depicts a side view of a plug structure for blocking an opening created in an intervertebral region, the plug structure having two tab structures to aid in its securement;

FIG. 18A depicts a cross-sectional transverse view of an intervertebral disc space having a intervertebral implant in a coiled configuration, the implant being contained by a covering sheet embodied as a laminate structure secured by securing devices attached to vertebrae;

FIG. 18B depicts a cross-sectional transverse view of an intervertebral disc space having a intervertebral implant, the implant being contained by a closing material and a covering sheet attached to vertebrae by securing pins;

FIG. 18C depicts a cross-sectional transverse view of an intervertebral disc space having a intervertebral implant, the implant being contained by a plug structure with tab structures for securing the plug structure to vertebrae with securing devices;

FIG. 19A depicts a cross-sectional transverse view of an intervertebral disc region having a nuclear pulposus extracted therefrom;

FIG. 19B depicts a cross-sectional transverse view of the disc region shown in FIG. 19A having a small intestine submucosa injectable formulation therein delivered by a delivery tube;

FIG. 19C depicts a cross-sectional transverse view of the disc region shown in FIG. 19B with the opening in the annular fibrosis closed by a gel formulation;

FIG. 19D depicts a cross-sectional transverse view of the disc region shown in FIG. 19B with the opening in the annular fibrosis closed by a plug structure held in place by pinned tab structures;

FIG. 20A depicts a cross-sectional transverse view of an intervertebral disc region within an annulus fibrosis having an intervertebral implant that includes a collapsible enclosure containing a filling material, the enclosure being sutured shut;

FIG. 20B depicts a cross-sectional axial view of the intervertebral disc region shown in FIG. 20A;

FIG. 21A depicts a side view of a herniated disc between two vertebral bodies;

FIG. 21B depicts a cross-sectional side view of the disc region shown in FIG. 21A having the herniated disc removed;

FIG. 21C depicts a cross-sectional side view of the cleared disc region of FIG. 211B with a collapsible enclosure being deposited from a delivery tube with a pusher, the enclosure having an expansion structure;

FIG. 21D depicts a cross-sectional side view of the collapsed enclosure shown in FIG. 21C filled with filling material and having the expansion structure oriented to allow asymmetric loading on the implant, the enclosure closed by a plug structure with tab structures and pins;

FIG. 22A depicts a perspective view of a collapsible enclosure with a collapsible support structure for use as an intervertebral implant;

FIG. 22B depicts a cross-sectional side view of a delivery tube containing a collapsible enclosure and collapsible support structure that can be advanced with a pusher;

FIG. 22C depicts a cross-sectional side view of an intervertebral implant at a disc replacement site, the support structure and enclosure adapted so the implant has a symmetric loading profile;

FIG. 22D depicts a cross-sectional side view of an intervertebral implant at a disc replacement site, the support structure and enclosure adapted so the implant has an asymmetric loading profile;

FIG. 23A depicts a cross-sectional axial view of a disc space having a support structure and enclosure deposited therein;

FIG. 23B depicts a cross-sectional axial view of the disc space shown in FIG. 23A with the enclosure expanded and the support structure rotated such that the opening of the support structure does not correspond with the opening in the annular fibrosis;

FIG. 24 depicts a perspective view of an enclosure and support structure emerging from the end of a delivery tube;

FIG. 25A depicts a side view of an intervertebral implant having a hybrid implant structure, the shaping structure of the hybrid structure tending to bias the implant toward a coiled configuration;

FIG. 25B depicts a side view of an intervertebral implant having a hybrid implant structure, the shaping structure of the hybrid structure tending to bias the implant toward a raveled configuration;

FIG. 25C depicts a side view of an intervertebral implant having a hybrid implant structure, the shaping structure of the hybrid structure tending to bias the implant toward a coiled configuration, the hybrid structure coiling around a core;

FIG. 26A depicts a perspective view of an intervertebral implant having a shaping structure embodied as a continuous layer between two small intestine submucosa layers;

FIG. 26B depicts a perspective view of an intervertebral implant having a shaping structure embodied as a strip;

FIG. 26C depicts a perspective view of an intervertebral implant having a shaping structure embodied as two embedded strips;

FIG. 27A depicts a cross-sectional transverse view of cleared intervertebral space and a delivery tube holding an intervertebral implant with a hybrid implant structure;

FIG. 27B depicts a cross-sectional axial view of the cleared intervertebral space shown in FIG. 27A;

FIG. 27C depicts a cross-sectional transverse view of the intervertebral implant of FIG. 27A partially delivered to the implantation site;

FIG. 27D depicts a cross-sectional axial view of the intervertebral implant shown in FIG. 27C;

FIG. 27E depicts a cross sectional transverse view of the intervertebral implant shown in FIG. 27A delivered to the implantation site;

FIG. 27F depicts a cross-sectional axial view of the intervertebral implant shown in FIG. 27E;

FIG. 28A depicts a perspective view of a reversibly deformable enclosure as a portion of an intervertebral implant having a shaping structure, the shaping structure adapted to bias the enclosure toward the shown configuration;

FIG. 28B depicts a close-up perspective view of the port of the reversibly deformable enclosure shown in FIG. 28A.

FIG. 28C depicts the reversibly deformable enclosure of FIG. 28A in a collapsed shape;

FIG. 29A depicts a cross-sectional side view of a reversibly deformable enclosure located within a delivery tube and being advanced by a pusher;

FIG. 29B depicts a cross-sectional side view of the reversibly deformable enclosure of FIG. 29A expanding after emerging from the delivery tube; and

FIG. 29C depicts a cross-sectional axial view of the reversibly deformable enclosure of FIG. 29A implanted within the space enclosed by an annulus fibrosis.

### DETAILED DESCRIPTION OF THE INVENTION

Certain exemplary embodiments will now be described to provide an overall understanding of the principles, structure, function, manufacture, and use of the devices and methods disclosed herein. One or more examples of these embodiments are illustrated in the accompanying drawings. Those skilled in the art will understand that the devices and methods specifically described herein and illustrated in the accompanying drawings are non-limiting exemplary embodiments and that the scope of the present invention is defined solely by the claims. The features illustrated or described in connection with one exemplary embodiment may be combined with features of other embodiments. For example, features, such as the types of tissue ingrowth enhancing materials that are described with reference to intervertebral implants having a tissue regeneration structure and a load bearing structure, can also be utilized with implant enclosures, implants that include a shaping structure, and injectable SIS formulations. Additionally and alternatively, one or more other features as understood by those skilled in the art can be combined with one or more features in an embodiment described herein. Such modifications and variations are intended to be included within the scope of the present invention.

Intervertebral Implants with a Tissue Regeneration Structure and a Load Bearing Structure

In one embodiment, an intervertebral implant includes a tissue regeneration structure and a load bearing structure that can be coupled together. The tissue regeneration structure, for promoting tissue growth, can include at least one layer of small intestine submucosa (herein “SIS”). The load bearing structure, for supporting loading on the intervertebral implant, can include a load bearing material. In general, the load bearing material can have a higher compressive modulus than the SIS when the implant is positioned within an intervertebral region of a patient. Before implantation, the SIS may be in a dehydrated, hardened state. After implantation, the SIS is typically hydrated, making the material more pliable and flexible. The SIS can also be hydrated before the implant is delivered to a patient.

SIS is a naturally occurring extracellular collagen based matrix. SIS is described in detail in U.S. Pat. No. 5,372,821, the disclosure of which is hereby incorporated by reference. As described in the '821 patent, SIS is a segment of intestinal tissue of a warm-blooded vertebrate, which segment comprises the tunica submucosa and basilar tissue of the tunica mucosa, where the tunica submucosa and basilar tissue are delaminated from the tunica muscularis and the luminal portion of the tunica mucosa of the segment of intestinal tissue. SIS contains cytokines and growth factors and has been shown to act as a resorbable scaffold in vivo that promotes soft tissue regeneration with little scar tissue formation. SIS can be manufactured in laminated sheets of various sizes and thicknesses for different indications.

When implanted into a subject, the tissue regeneration structure (e.g., the SIS layer) can serve to promote intervertebral tissue growth and integration of the implant into the spinal region. The load bearing structure can serve to help bear the loads that the implant is subject to when implanted in the intervertebral region. In particular, the load bearing structure can act to improve the load bearing and/or shape retention characteristics of the implant relative to devices manufactured solely with SIS, or similar materials, that are more flexible and that may be more subject to deformation after extended use. For instance, exemplary implants can bear a greater load before deforming to a particular degree. In another instance, the tissue regeneration structure is coupled to a shape retaining structure, the shape retaining structure being more resistant to shape change under intervertebral loading in a patient than the tissue regeneration structure. Thus, the shape retaining structure can help the implant retain a desired geometric configuration (e.g., a desired disc height) under a particular static load or after being subjected to a particular cyclical loading profile. It is understood that the materials and geometries of a load bearing structure described herein can also be applied to shape retaining structures, so long as the shape retaining structure is more resistant to shape change than the tissue regeneration structure.

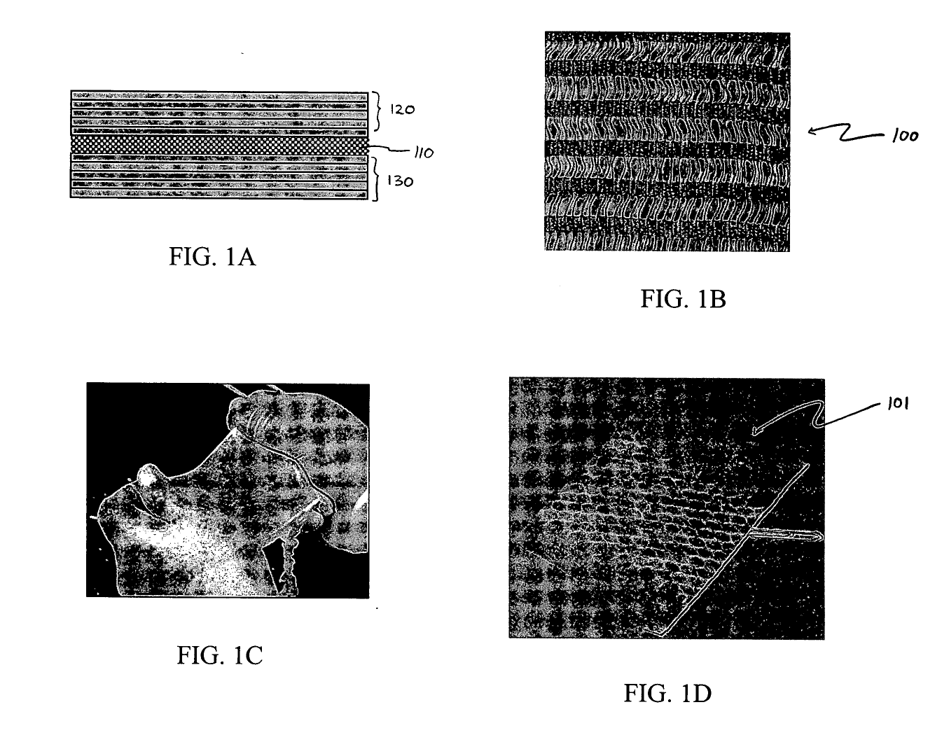

In general, tissue regeneration structures can be formulated with any combination of materials that can act as a scaffold to help promote tissue replacement, repair, and/or regeneration. SIS layers that are utilized as part of a tissue regeneration structure can utilize SIS from any, or a combination of, sources (e.g., bovine or porcine). Typical SIS layers can be formed by stretching a portion of small intestine submucosa into a layer or sheet-like structure as shown in FIG. 1C. SIS layers can utilize any shape or size necessary to form an effective tissue regeneration structure. For example, SIS strips can be utilized having a length of about 5 to about 1000 millimeters, a width of about 0.5 to about 50 millimeters, and a thickness of about 0.1 to about 2 millimeters. Beyond the use of SIS, tissue regeneration structures can include other types of resorbable materials such as other types of extracellular materials (herein “ECMs”). The load bearing structure (or shape retaining structure) utilizes materials suitable to perform the various functions described herein. Suitable materials include both resorbable and non-resorbable materials.

Types of resorbable materials that can be utilized with various embodiments include materials that are ECM-, ceramic-, and/or polymer-based. These include, but are not limited to, autograft/allograft/xenograft tissues (e.g., SIS, pericardium, acellular dermis, amniotic membrane tissue, cadaveric fascia, bladder acellular matrix graft, etc.), collagen, hyaluronic acid, elastin, albumin, silk, reticulin, prolamines, polysaccharides, alginate, plasmin, thrombin, fibrin, heparin, hydroxyapatite, tri-calcium phosphate, tri-calcium sulfate, calcium sulfide, glues/adhesives, cyanoacrylates, crosslinking agents (e.g., formaldehyde, gluteraldehyde, albumin, etc.), biodegradable polymers of sugar units, synthetic polymers including polylactide, polyglycolide, polydioxanone, polyhydroxybutyrate, polyhydroxyvalerate, poly(propylene fumarate), polyoxaesters, polyesters, polyanhydrides, tyrosine-derived polycarbonates, polyorthoesters, polyphosphazenes, synthetic polyamino acids, biodegradable polyurethanes and their copolymers, and any combination of the aforementioned materials.

Types of non-resorbable materials that can be utilized with various embodiments include materials that are metallic-, ECM-, ceramic-, and/or polymer-based. These include, but are not limited to, autograft/allograft/xenograft tissues in crosslinked forms (e.g., SIS, pericardium, acellular dermis, amniotic membrane tissue, cadaveric fascia, bladder acellular matrix graft, etc.), polyacrylates, ethylene-vinyl acetates (and other acyl-substituted cellulose acetates), polyester (e.g., Dacron®), poly(ethylene terephthalate), polypropylene, polyethylene, polyurethanes, polystyrenes, polyvinyl oxides, polyvinyl fluorides, poly(vinyl imidazoles), chlorosulphonated polyolefins, polyethylene oxides, polyvinyl alcohols (PVA), polytetrafluoroethylenes, nylons, silicones, polycarbonates, polyetheretherketones (PEEK) with or without carbon fiber reinforcement, stainless steel alloys, titanium alloys, cobalt chromium alloys, and combinations of the aforementioned materials.

Implants consistent with the exemplary embodiment can be utilized in a variety of manners to treat intervertebral ailments. In several embodiments disclosed herein, the implant can be used as a prosthesis to replace a portion, or an entire, nuclear pulposus of an injured disc. Such implants can also serve to replace a portion, or an entire, annulus fibrosis (e.g., an opening in an annulus for inserting a nuclear pulposus prosthesis), or can act as an entire disc replacement prosthesis. The implants, however, can also be used to replace various other intervertebral structures including spinal ligaments (e.g., annular longitudinal ligament, posterior longitudinal ligament, posterior interspinous ligament), facets (e.g., facet joint), and combinations thereof. The implants can also be used to block an opening formed in an intervertebral region, or can act as a securing device (e.g., a screw or suture). Specific embodiments discussed herein provide non-limiting examples of uses of particular types of implants.

The shape, size, and orientation of the load bearing structure and the tissue regeneration structure can be any that achieves the desired functionality of the implant. The load bearing structure or the tissue regeneration structure can be an integral body, or adapted to be a plurality of separate bodies. The load bearing structure and tissue regeneration structure can be a plurality of intertwined bodies (e.g., a weave of SIS strips with load bearing material strips to form a layer that can be folded or coiled), or may be individual, integral structures that are coupled together. As well, implants can generally be pre-fabricated to have a particular shape that is retained at an implantation site, or the implant can be shaped after the implant is delivered to the implantation site. Though particular embodiments are described herein, these merely serve to exemplify some of the potential implant structures that are within the scope of the invention.

FIG. 1A depicts an embodiment of an implant in the form of a laminate structure. The tissue regeneration structure is embodied as two sets 120, 130 of four stacked SIS layers that contact the top and bottom surface of a load bearing structure 110 embodied as a layer of load bearing material. In one embodiment, the load bearing structure 110 can be a polydioxanone (herein “PDO”) mesh having pores (e.g., about 4 mm as shown in FIG. 1B). The PDO mesh 100 shown in FIG. 1B includes polydioxanone sutures (herein “PDS”), the mesh having a burst strength of about 800 newtons, which provides substantial load bearing properties to the implant. A photograph of a manufactured implant 101 is shown in FIG. 1D.

When a mesh structure is utilized as the load bearing structure, the structure can be impregnated with materials that enhance the functionality of the implant. For example, one or more bioactive factors can be utilized to improve tissue ingrowth into the implant and/or provide other advantageous functions. Suitable bioactive factors include but are not limited to platelet-rich plasma, platelet-poor plasma, bone marrow aspirate, whole blood, serum, transforming growth factor-beta and agents in the same family of growth factors (e.g., TGF-131, TGF-1132, and TGF-133, GDF-5, MP52, and/or bone morphogenetic proteins), platelet-derived growth factors, fibroblast growth factors, insulin-like growth factors, vascular endothelial growth factors, tumor necrosis factors, interleukins (e.g., IL-1, IL-6, etc.), prostaglandins, protein polymers such as RGD-peptides, PHSRN-peptides, YIGSR-peptides and Indian Hedgehog proteins, ECM proteins/components (e.g. fibronectin, laminin, thrombospondin, glycosaminoglycans, proteoglycans, etc.), anti-inflammatory agents, anti-microbial agents, anti-catabolic agents, anabolic agents, drugs, pharmaceutical agents, viral and nonviral vectors, DNA, RNA, angiogenic factors, hormones, cells, enzymes, hyaluronic acid and the like. In another example, one or more particular cell types can be added in a supporting medium (optional) to entrain the mesh to promote ingrowth. Examples of cells include cells harvested from spinal discs such as nucleus pulposus cells and annulus fibrosis cells and endplate cells. Other examples include but are not limited to stem cells (embryonic and adult), bone marrow cells, osteogenic, fibrogenic, adipogenic, myogenic, and/or chondrogenic cells. Though the use of biofactors and cells are discussed with reference to impregnating the mesh of a load bearing material, such factors and cells can also be entrained within the tissue regeneration structure or be another portion of the implant (e.g., a separate layer of material in a laminate structure or be used to fill a small volume in the implant).

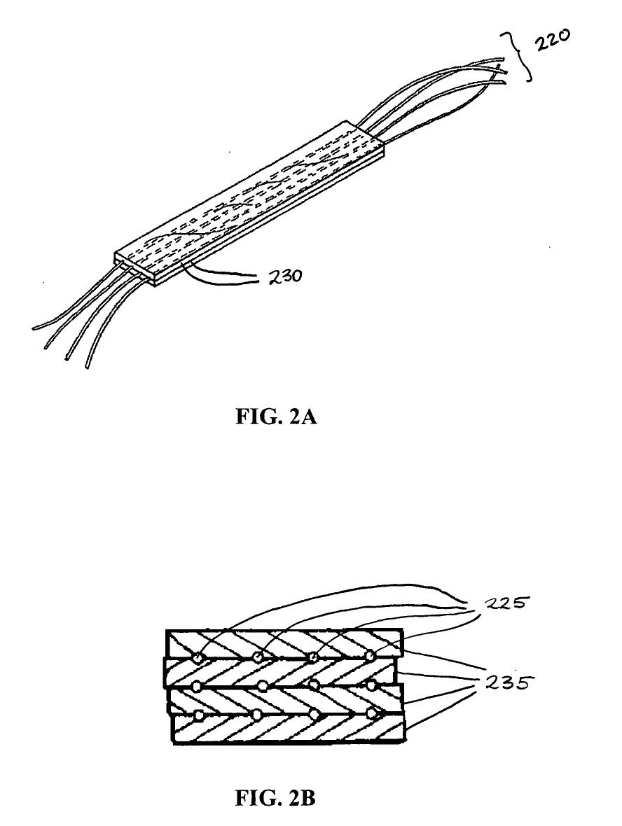

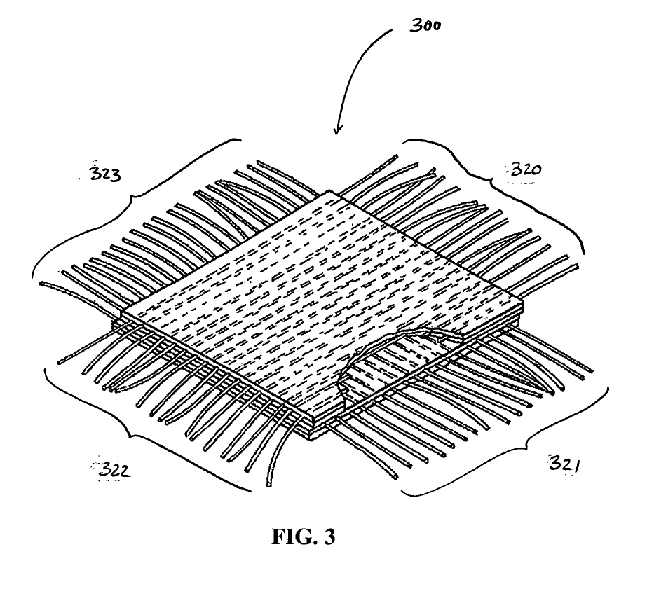

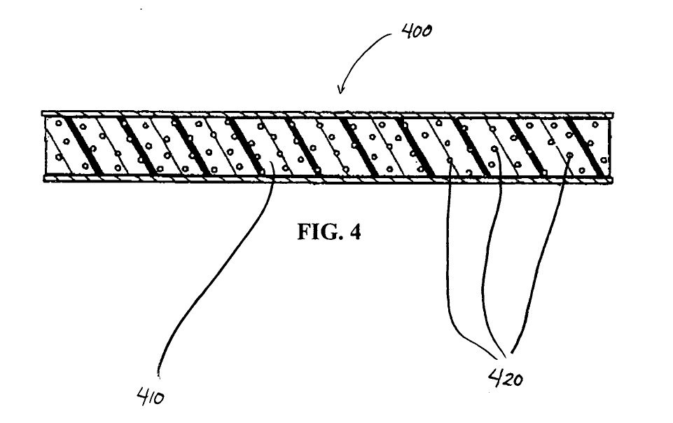

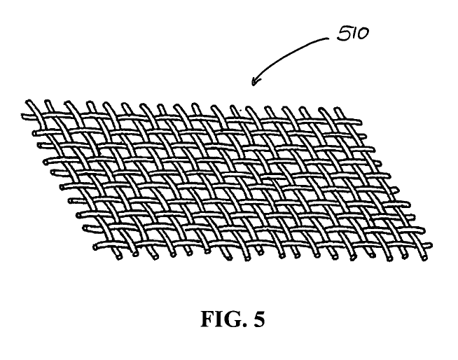

The laminate structure shown in FIG. 1A utilizes a plurality of layers of material, each layer being embodied as a finite continuous layer. In general, however, a layer can also be formulated as a non continuous structure. For example, a layer can be embodied as one or more elongate structures, such as a plurality of fibers 220, 225 that are sandwiched by continuous layers 230, 235 as shown in FIGS. 2A and 2B. In another example, an implant 300 includes sets of fibers that can act as one or more layers 320, 321, 322, 323, the sets of fibers being oriented in more than one direction as shown in FIG. 3. As shown in the cross sectional view of FIG. 2B, the fibers 225 can be embedded between layers 235 of material. Alternatively, the fibers 420 can be embedded in a matrix 410 as depicted in the cross sectional view of the implant 400 depicted in FIG. 4. Fibers can also be woven into a mesh structure 510 as depicted in FIG. 5. Meshes can optionally be embedded in another material. Clearly, other elongate structures besides fibers can be utilized (e.g., strips or beam-like structures or combinations of the various elongate structures).

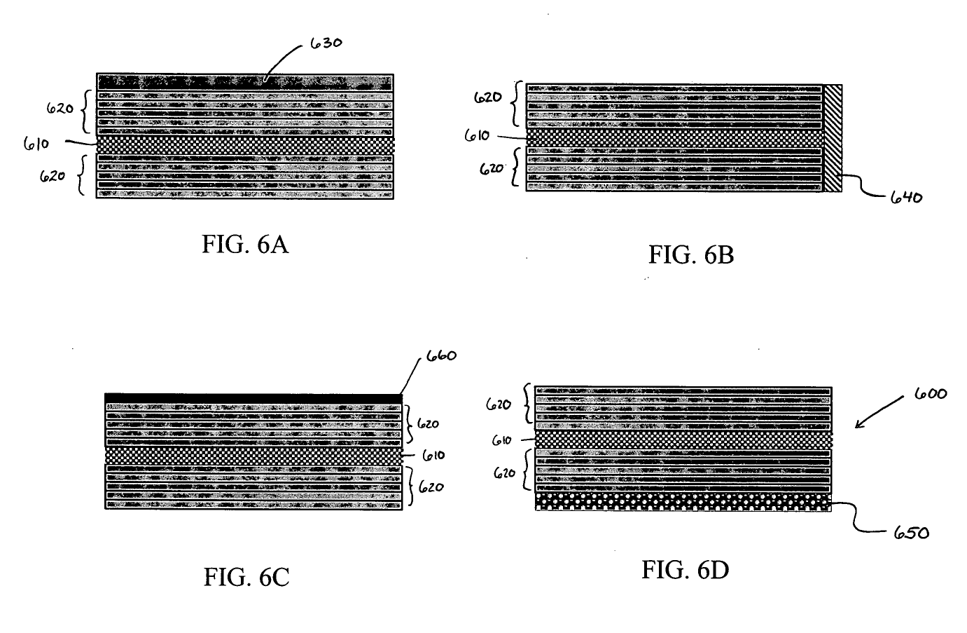

The layers of a laminate structure can be held together applying a compatible adhesive (e.g., cyanoacrylate) between the layers of the structure. Layers of a load bearing material 610 and a tissue regeneration structure 620 can also be held together using an end layer 640 that is oriented substantially perpendicular to the direction of the layer surfaces and attached to their ends as depicted in FIG. 6B. The end layer 640 can optionally include SIS to enhance the ability of the implant to integrate within the body (e.g., integrate with the annulus fibrosis, or to act like a cartilaginous end plate). In such an instance, the implant can be positioned such that the end layer contacts an intended tissue surface. End layers can also include cells and/or bioactive factors, as discussed herein, for delivery to adjacent tissues.

Other layers can also be incorporated into a laminate structure to provide further functionality. For example, a layer containing tissue ingrowth enhancing components, such as any combination of the biofactors and/or cells previously discussed, can be included. In particular, the biofactors and/or cells can be encapsulated in spheres or other particulates that degrade upon pressure contact or exposure to heat, electrical current, ultrasound waves, and/or radiation (e.g., UV or visible light), allowing release of the tissue ingrowth components. In another example, a layer 630 that contains osteoinductive materials can be included with load bearing material layer 610 and layers 620 forming a tissue regeneration structure, an example being depicted in FIG. 6A. Such materials can promote bony tissue growth into the implant and/or promote attachment of implant to the intervertebral region of the body. An osteoinductive material can include, but is not limited to, one or more of the following materials: platelet rich plasma, bone barrow, stem cells, osteogenic cells (e.g., osteoblasts), demineralized bone matrix, bone morphogenic protein (e.g., MP52), growth factors, hydroxyapatite, hyaluronic acid, calcium phosphate, and calcium sulfate. The osteoinductive material can also be formulated to stimulate fusion of the implant in the intervertebral region.

Additional layers can also include materials that act as an adhesive layer or an anti-adhesion layer as depicted in FIGS. 6D and 6C, respectively. The adhesive layer 650 utilizes compatible materials to adhere the implant 600 to tissue (e.g., bone, the cartilaginous endplate, or the annulus fibrosis). The adhesive layer 650 can also include tissue ingrowth materials to promote prosthesis attachment to the body. The adhesive and/or tissue ingrowth materials can be encapsulated in particulates to allow release of the agents upon pressure contact or exposure to heat, electrical current, ultrasound waves, and/or radiation (e.g., UV or visible light). Anti-adhesion layers 660 (e.g., a layer including hyaluronic acid) can be used to prevent a portion, or the entire, implant from inadvertently adhering to tissue and/or other structure with an implantation site.

Though various materials described herein are distributed as layers in an intervertebral structure, it is clear that such materials can also be distributed in other manners within, on, or around an intervertebral implant. Materials such as biofactors, osteoinductive materials, cells, adhesives, and other tissue ingrowth enhancing components can be distributed as coatings on portions of an implant. For example, an adhesive can coat a portion of an implant that is adapted to contact tissue. Various materials can also be disposed in interstices formed within, or between, structures of the implant. Examples include a gap formed between layers of a laminate structure or a hollowed region in a load bearing block structure of an implant. One skilled in the art will readily appreciate the range of manners in which such materials can be incorporated with an intervertebral implant.

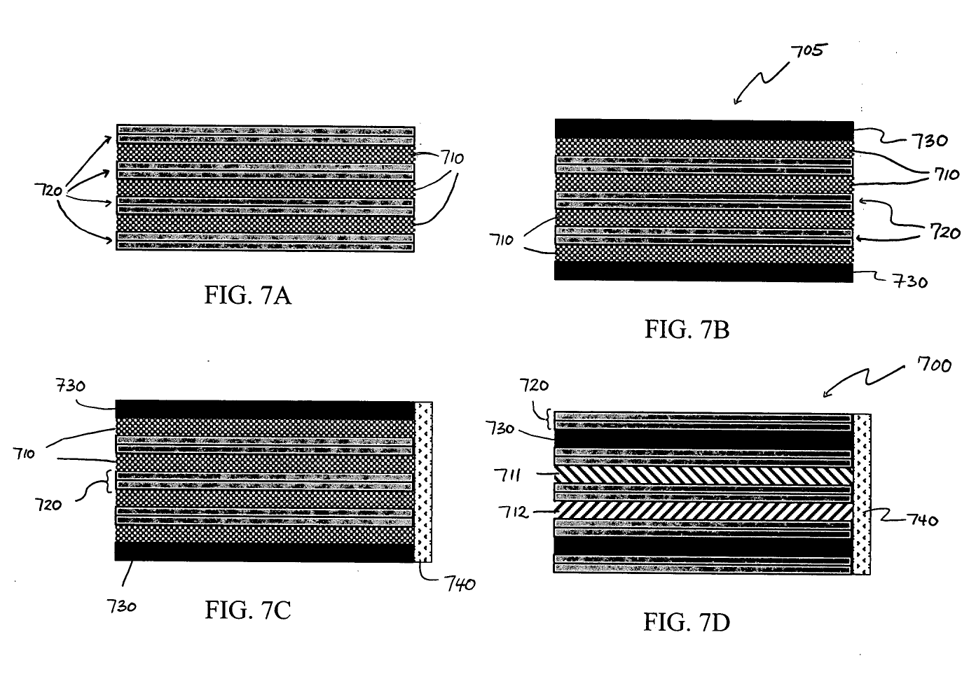

Though the laminate structures shown in FIGS. 1A and 6A-6D utilize a specific number of layers for the tissue regeneration and the load bearing structures, any number and type of layers can be used and arranged for portions of a laminate structure. FIGS. 7A-7D illustrate further embodiments of a laminate structure that can act as intervertebral laminates. FIG. 7A depicts multiple dual layers 720 of SIS acting as a tissue regeneration structure interrupted by load bearing material layers 710 that can act as the load bearing structure. FIG. 7B replaces the top SIS layers 720 with layers 730 that include tissue ingrowth materials. FIG. 7C includes an end layer 740 with the implant 705 of FIG. 7B. FIG. 7D depicts another implant 700, including layers 730 of tissue ingrowth materials and layers 720 of a tissue regeneration structure, in which the load bearing layers 711, 712 each have particulates with a chosen orientation in the respective layer 711, 712. Such orientation of particulates can increase the load bearing properties of the implant. For example, fibers can be substantially oriented in layer 711 in one particular direction. The fibers in layer 712 can be oriented substantially in another direction not aligned with the fibers of the first layer 711 (e.g., perpendicular). The combination of layers 711, 712 thus act to strengthen the load bearing properties of the entire prosthesis. Since SIS layers can also be comprised of SIS fibers that are oriented in a particular direction, SIS layers can also be positioned such that two or more layers have fibers that are not aligned to thereby increase the strength of the ensemble of SIS layers.

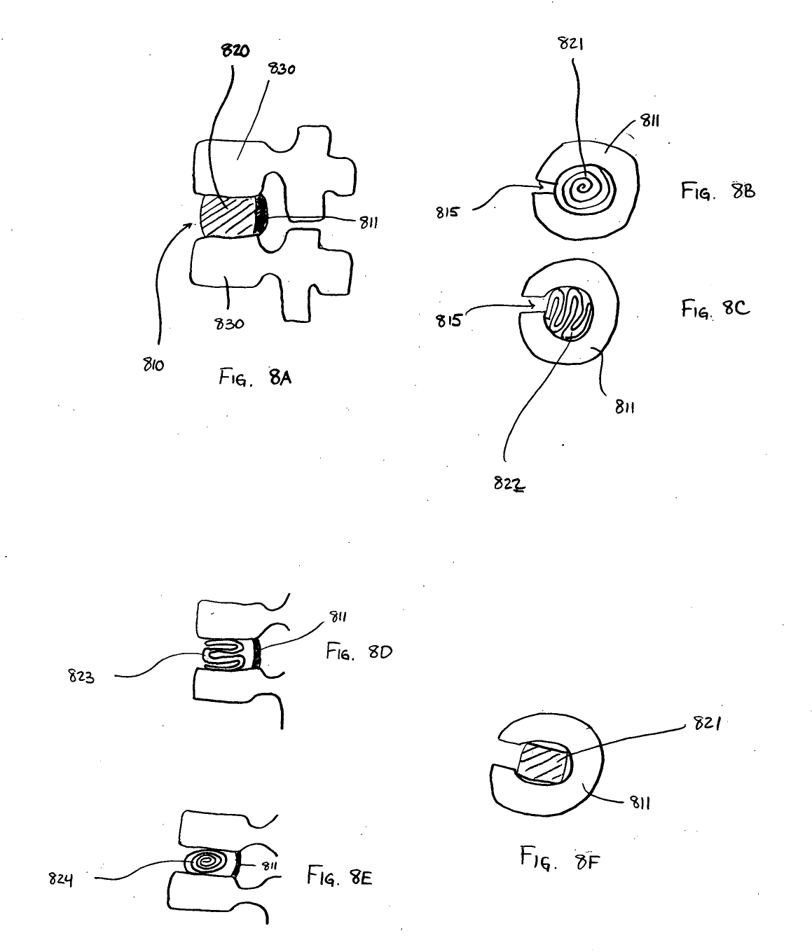

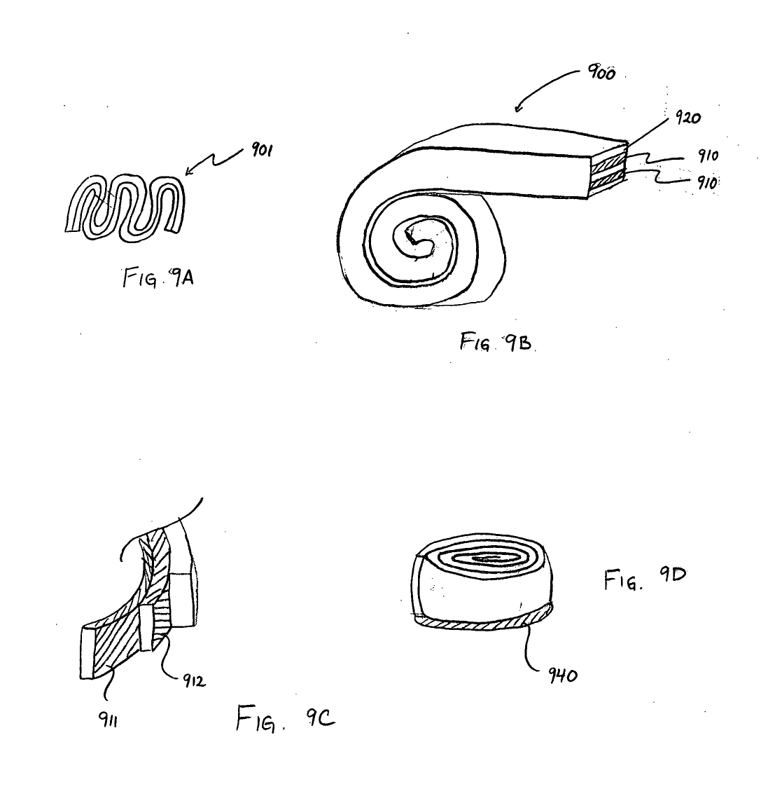

Intervertebral implants, including laminate structures, can be folded or otherwise configured into an implantable structure that is adapted to fit into at least a portion of an intervertebral space. FIG. 8A presents a transverse view of a space 810 between two vertebrae 830 that is partially filled by an exemplary folded intervertebral implant 820. FIGS. 8B and 8C present axial views of particularly configured implants 821, 822 surrounded by an annular fibrosis 811, the space 815 in the annular fibrosis 811 corresponding to the space 810 shown in the transverse view of FIG. 8A. The particular shape of the folded structure 820 can include a variety of configurations such as a simple alternated folded laminate configuration 822, 823, 901 as depicted in FIGS. 8C, 8D, and 9A or a coil-like configuration 821, 824, 900 as depicted in FIGS. 8B, 8E, and 9B. The implant can also be positioned in a variety of orientations. For example, FIGS. 8B and 8C depict the axis of the coil configuration 821 and the folds of the folded configuration 822 oriented substantially parallel the direction of the axis of the vertebrae. In contrast, the transverse views of FIGS. 8D and 8E depict the axis of the coil configuration 824 and the folds of the folded configuration 823 oriented substantially perpendicular to the axis of the vertebrae. FIG. 8F presents an exemplary axial view of an implant 821 in an annular fibrosis 811 that can correspond with the transverse views presented in FIGS. 8D and 8E. As exemplified in FIG. 9B, the implant 900 can comprise plural layers of SIS 910 and load bearing layers 920 that form a laminate structure that can be coiled together. As discussed earlier, SIS layers or load bearing layers can be oriented such that fibers in one layer 911 are not oriented with respect to the fibers in another layer 912 to provide greater implant strength as depicted in FIG. 9C. A folded implantable structure can also include one or more additional layers (e.g., a layer of tissue ingrowth materials such as biofactors and/or cells, a layer of adhesive or an anti-adhesive, or a layer having a combination of the aforementioned components) that can be aligned as part of a laminate structure, or as an end layer 940 having a surface oriented substantially perpendicular to surfaces of the laminate layers as exemplified by the structure depicted in FIG. 9D.

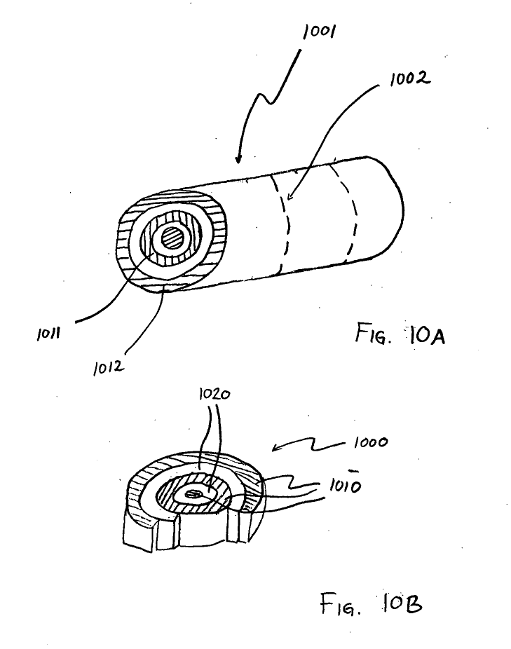

Another configuration for an implantable prosthesis can include a plurality of nested bands as exemplified in FIGS. 10A and 10B. The implant 1000 can be formed from alternating nested bands of SIS material 1010, forming the tissue regeneration structure, and load bearing material 1020 forming the load bearing structure as shown in FIG. 10B. The implants can also be formed from a roll of nested bands 1001 that are cut into pieces (as indicated by the dotted lines 1002 in FIG. 10A). As previously described, other layers of material can be added, as bands or as an end layer, to provide tissue ingrowth properties, adhesion, anti-adhesion and other functionality. As well, SIS and non-SIS layers can also be provided with oriented fibers. For example, as shown in FIG. 10A, one layer 1011 can have fibers that are oriented in a different direction than the fibers of another layer 1012.

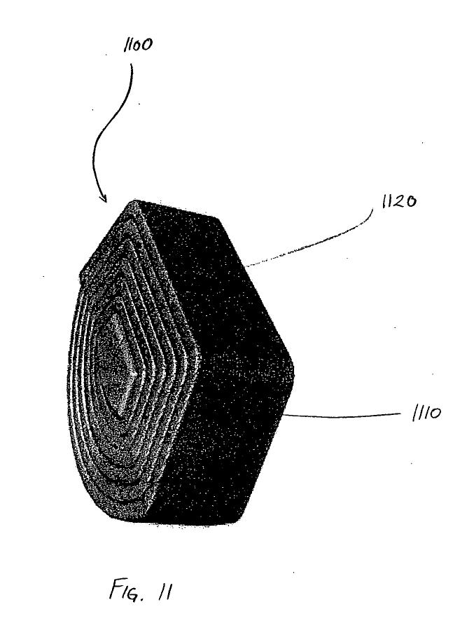

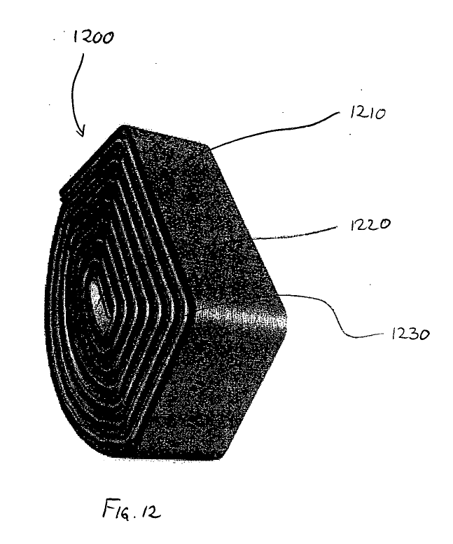

FIG. 11 depicts another embodiment of an intervertebral implant. The implant 1100 includes a tissue regeneration structure that is formed as a multilayered structure with adjacently located surfaces. The load bearing structure can be formulated as one or more block structures that can be embedded within the multilayered structure. In the embodiment illustrated in FIG. 11, the block structure is a core 1120 that can be located substantially in the center of the implant 1100 with a wrapped multilayered structure 1110 positioned around the core 1120 (i.e., coiled). In another embodiment, as shown in FIG. 12, the implant 1200 has a load bearing structure that includes the core 1220 and at least one layer 1230 of load bearing material. The layers of load bearing material 1230 are positioned so as to be adjacent to a tissue regeneration structure embodied as one or more layers of SIS 1210, and wrapped around core 1220. Other arrangements of layers for the load bearing structure and the tissue regeneration structure can also be utilized, including any of the laminate structures shown in FIGS. 6A-6D and 7A-7D which can be wrapped around a core. One or more of the other types of layers described for laminate structures can also be incorporated with the wrapped laminate, one example being an end layer such as the layer 940 depicted in FIG. 9D.

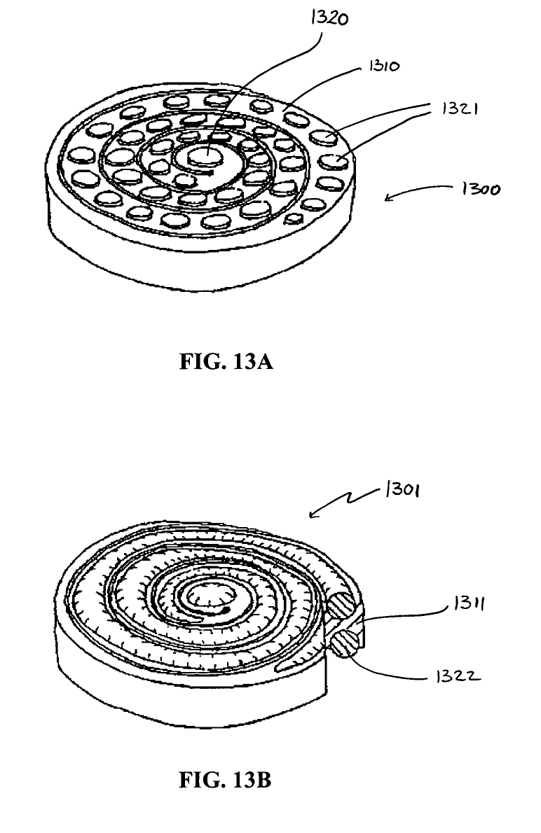

Block structures as utilized in an intervertebral implant can include any materials that are appropriate for a load bearing structure. Block structures can also be configured and arranged in a variety of geometries to facilitate the implant's load bearing capacity. As shown in FIG. 13A, implant 1300 has a plurality of block structures 1321 that can be embedded within a tissue regeneration structure 1310 (or between layers of SIS). Though the implant 1300 is shown to include a core 1320, the presence of a core is optional. Block structures can also be oriented in the coiling direction as shown by the coiled elongates 1322 embedded in material 1311 of implant 1301 shown in FIG. 13B.

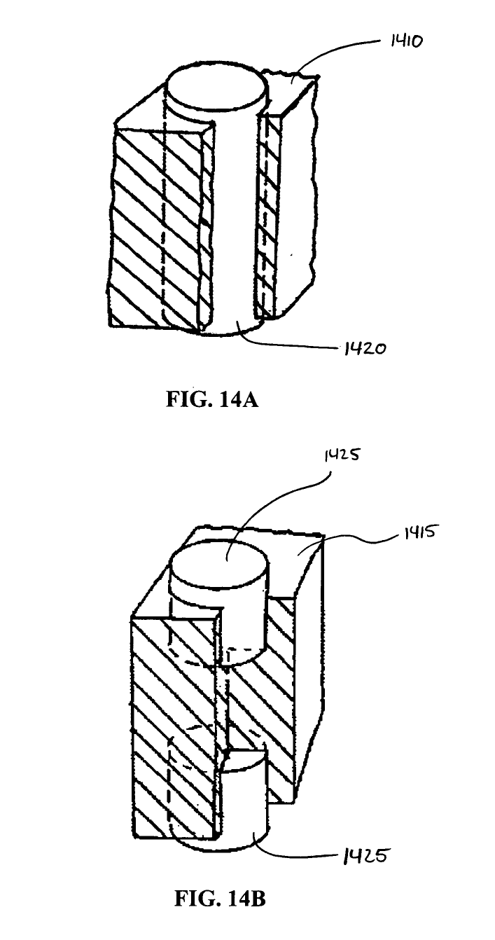

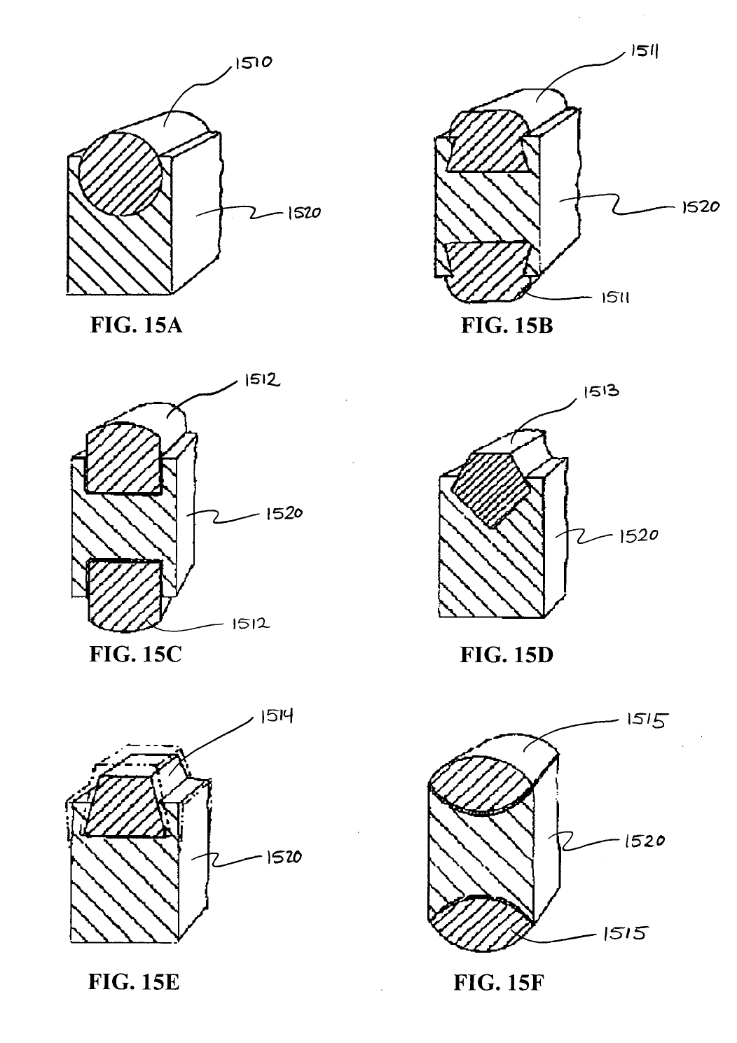

Block structures can also be configured to penetrate through an entire width 1410 of an implant using a pillar-like configuration 1420 as shown in FIG. 14A, or the structures 1425 can only partially penetrate an implant width 1415 as shown in FIG. 14B. Block structures 1510, 1511, 1512, 1513, 1514, 1515 can also be assembled in any number of shapes, some examples being shown in FIGS. 15A-15F. Matrix material 1520 can have a specific cut-out cross-section to accommodate the specific shape of a block structure 1510, 1511, 1512, 1513, 1514, 1515. Alternatively, the block structures can be positioned between composite layers of material, or act as a core in which a laminate structure is wrapped around.

In another exemplary embodiment, one or more block structures can be incorporated into a plurality of nested bands, creating a modification to the structure shown in FIGS. 10A and 10B. Such blocks can be incorporated as cores, pillars, partially penetrating blocks, or elongate encircling structures within the nested band implant.

Other embodiments can utilize one or more block structures as a portion of a tissue regeneration structure. For example, the core 1120, 1220 shown in FIGS. 11 and 12 can be part of a tissue regeneration structure (e.g., including arrangements of SIS or other resorbable materials), the load bearing structure being laminates or some other block structure wrapped around the core. Clearly, blocks in the form of pillars, elongate coils, or other configurations can similarly act as at least a portion of a tissue regeneration structure. Blocks, acting as a portion of a tissue regeneration structure or a load bearing structure, can also be formed to have a porous nature, or include a pocket area, to hold additional materials such as biofactors and/or cells to facilitate the incorporation of the implant into a patient's body.

Intervertebral implant structures with a tissue regeneration structure and a load bearing structure can be adapted to deform asymmetrically under a uniform load. By having a preferred loading direction, or deformation direction, implants can aid in correcting abnormalities is spinal anatomy (e.g., lordosis or kyphosis). For example, for the implant depicted in FIG. 13A, the block structures on one section of the implant can have a higher modulus than the block structures on the remaining portions of the implant. As such, the implant can be more supportive in one direction and more pliant in another. In another example, load bearing block structures having larger cross-sectional areas can be utilized in one section of an implant, while load bearing block structures with smaller cross-sectional areas are utilized in another section. If the number density of block structures in the implant is uniform, the section with larger cross section block structures can sustain more loading than the section with small cross-section block structures. Those skilled in the art will recognize that a number of modifications can be made to the embodiments of an implant having a load bearing structure and a tissue regeneration structure to allow implant loading in a preferred manner.

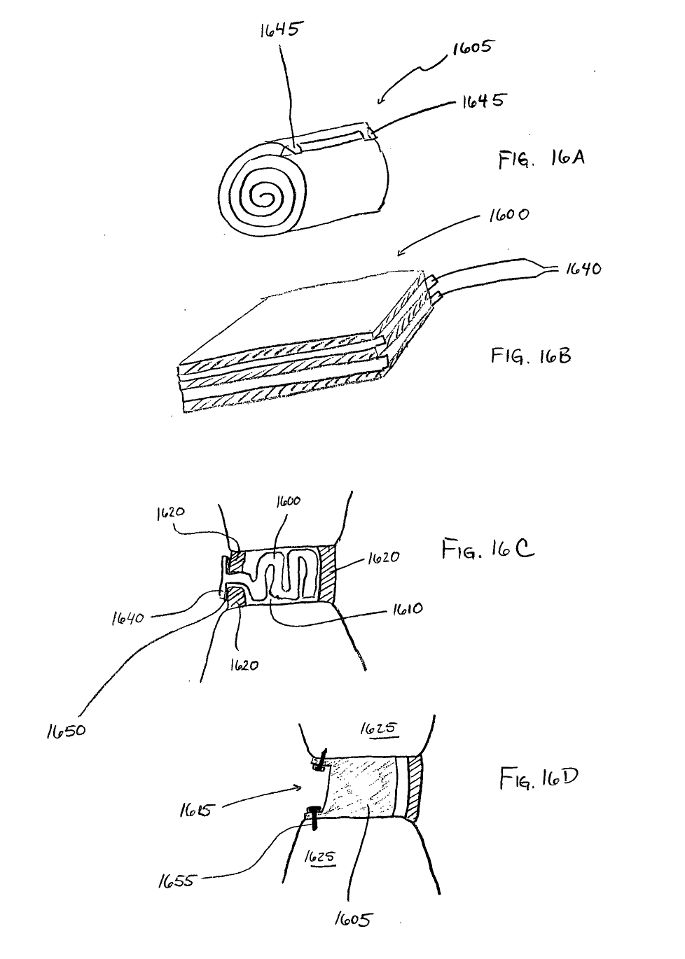

Implant structures, as described herein, can include one or more tab structures that extend from a portion of the implant to help secure the implant upon implantation. As depicted in FIG. 16A, an implant 1605 includes tab structures 1645 that extend from one end of the rolled laminate. Another example is provided by the tab structures 1640 extending from the laminate structure 1600 shown in FIG. 16B. When an implant is inserted into an intervertebral space, tab structures can be used to secure the implant to tissue. Accordingly, an implant 1600, 1605 can be secured in a disc space 1605, 1615 by attachment to bone 1625 or to soft tissue such as an annulus fibrosis 1620 as depicted in FIGS. 16C and 16D. Though implant structures generally do not necessarily require attachment to tissue, such attachment can help prevent or mitigate prosthetic migration and/or expulsion from a desired implanted location. Any number of mechanisms can be used to secure the tab structure to tissue, one example being the use of securing devices such as a screw or tack 1655 as shown in FIG. 16D. Adhesive compositions 1650 or other attachment devices and compositions can also be used. In one embodiment, a tab structure can include SIS and/or other types of resorbable material to facilitate integration with the tissue to which the implant is attached. It is also understood that, in general, an implant can be attached to tissue without the use of a tab structure (e.g., by direct suturing of the implant to tissue).

Tab structures can be shaped and sized in a variety of manners to be effective to receive one or more securing devices to attach the implant. Accordingly, the tab can be an elongated flap or a skirt-like flap that can be secured with a variety of securing devices including pins, screws, and tacks. Tab structures can also be a separate structure from the tissue regeneration structure and load bearing structure. For example, a sleeve can be fitted over a portion or the entirety of the tissue regeneration structure and load bearing structure, the sleeve including the tab structures for attachment of the implant. Optionally, tab structures may be part of the load bearing and/or tissue regeneration structures, the tabs being covered by an outer sleeve. Slits in the sleeve can be positioned to allow tab structures to extend out (i.e., the tabs can be retracting or telescopic) from the slit after the implant is positioned in a patient.

In another exemplary embodiment, an implant can include one or more securing devices effective to attach the implant to tissue (e.g., bone or soft tissue). Types of securing devices include screws, sutures, pins, tacks, nails, staples, and other fastening devices. In particular, the securing device can be constructed of materials including SIS and/or other resorbable materials to facilitate the device integration with the tissue. The SIS and/or other resorbable materials can constitute the whole of the securing device, or they can be a portion of the securing device.

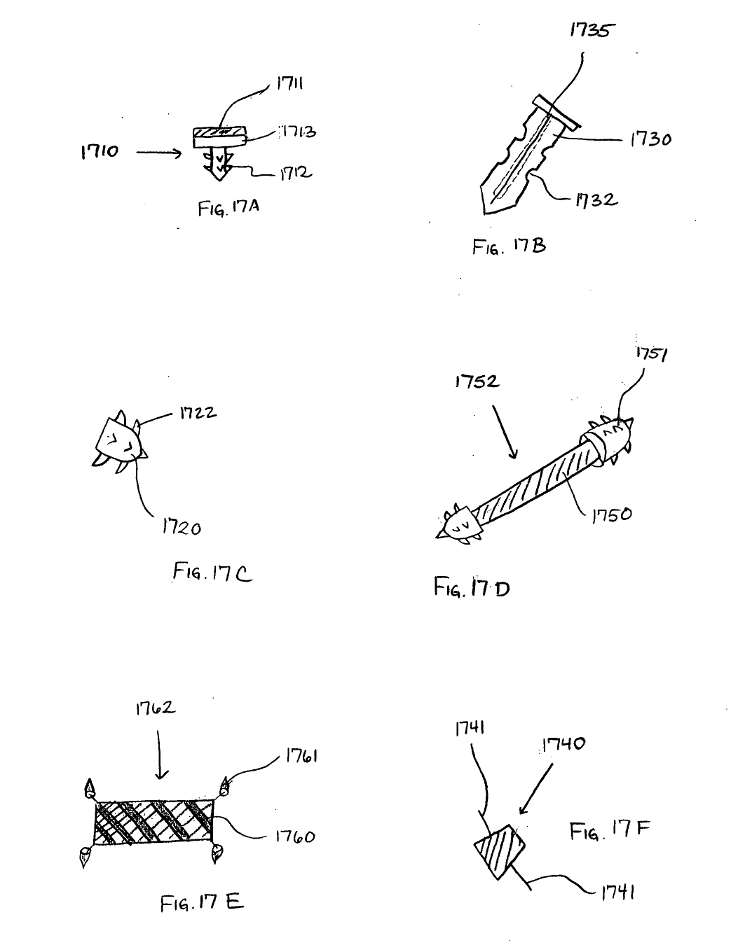

Various geometries that can be useful with securing devices 1710, 1720, 1730 are depicted in FIGS. 17A-17C. The specific securing device 1710 of FIG. 17A can include a layer 1711 of resorbable material on the head 1713 of the device 1710 to help integration of the device 1710 with a patient's body. An internal volume 1735 can be positioned within a securing device 1730, as depicted in FIG. 17B, for holding materials such as biofactors, cells, and/or other tissue ingrowth enhancing materials to enhance the ability of the securing device 1730 to integrate with the contacted tissue. Securing devices 1710, 1720, 1730 can optionally include structures 1712, 1722, 1732 such as threads, burrs, or spars to facilitate attachment to tissue. Any combination of these features, and others as understood by those skilled in the art, can be utilized with securing devices.

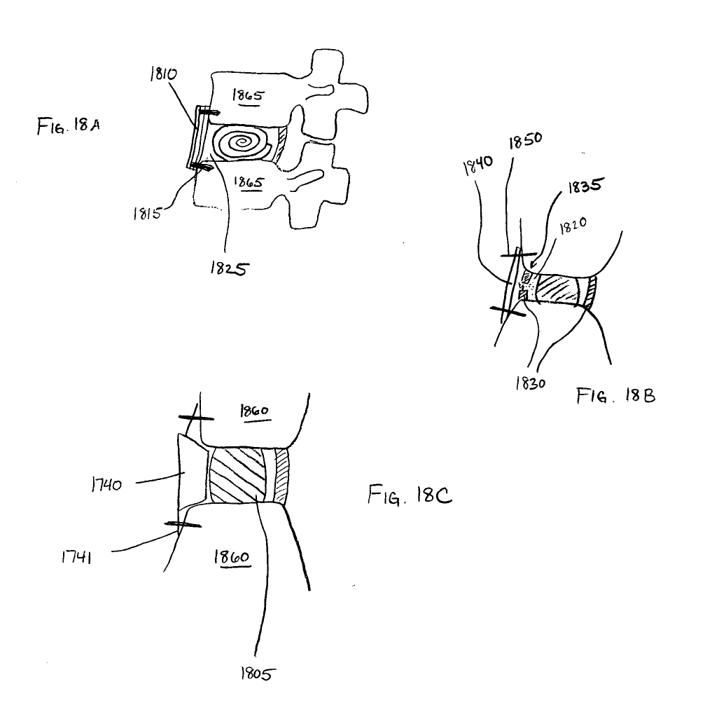

Though many of the embodiments discussed herein refer to the implant acting as a prosthetic device to replace at least a portion of an intervertebral disc, other exemplary embodiments are directed to implants that are plug-like structures that cover or occlude an opening to a space in an intervertebral region (e.g., a disc space). Accordingly, such implants can include a tissue regeneration structure and a load bearing structure in accord with embodiments described earlier. The space can include a prosthetic device, and/or have resident tissue which has been treated for an ailment. One exemplary embodiment, depicted in FIG. 17F, is a plug structure 1740 that is formed from a tissue regeneration structure and a load bearing structure as previously described. The plug structure 1740 can include tab structures 1741 for aiding attachment of the plug structure 1740 to tissue 1860 to hinder potential expulsion of a implant 1805 from a disc space, as depicted in FIG. 18C. It is understood that the plug structures need not include tab structures to assist in attaching or securing the plug. Instead, the plug structure can be held in place by adhesive located between the plug structure and tissue.

In another embodiment, prosthetic implants can also include a device to cover or block an opening to a space in an intervertebral region (e.g., a disc space). Unlike some embodiments where an implant includes a load bearing structure and a tissue regeneration structure, the blocking devices can be created from any combination of materials that are suitable for implantation and effective to close or seal the opening. Accordingly, the covering or blocking devices can include resorbable and/or non-resorbable materials, and can also include embodiments that have both a load bearing structure and tissue regeneration structure. Examples of the geometries of such devices are depicted in FIGS. 17D-17F. The devices 1752, 1762 shown in FIGS. 17D-17E include a plurality of securing devices 1751, 1761 that are coupled to a covering layer 1750, 1760. Covering layers can include one or more sheets of resorbable material (such as a plurality of SIS sheets) or can be a weave of resorbable and non-resorbable materials as depicted in FIG. 17E. The securing devices that are coupled to a covering layer can be utilize as shown in FIG. 18A in which the covering layer 1810 is oriented to cover an opening 1825 while securing devices 1815 are embedded in tissue (e.g., bone 1865) to hold the covering layer 1810 in place.

Another example of a closing or blocking device is depicted in FIG. 18B in which closing material 1820 is positioned in an intervertebral space to close an opening 1835 in an annulus fibrosis 1830. The closing material can have a flowable, or paste-like consistency upon application to the opening, and it should be capable of subsequently aging to a more hardened state. Optionally, as shown in FIG. 18B, a covering sheet 1840, held in place by securing devices 1850, can also be used to cover the opening. Plugs and other blocking devices can also be utilized in conjunction with the closing material. Appropriate closing materials include the variety of compositions which are compatible for use in filling the intervertebral region. Components of the closing material can include one or more of hydrogels, resorbable or non-resorbable polymers, thrombin or other clotting agents, bone wax, bone cement, cross linking agents, annular fibrosis tissue, and intervertebral disc cells with or without a carrier. Some closing materials can require the subsequent application of pressure contact, heat, electrical current, ultrasound waves, and/or radiation exposure (e.g., UV and/or visible light) after the closing material is applied to an opening to effect a change in their hardness.

Delivery of these intervertebral implants to damaged or diseased regions of a spine are well within the knowledge of those skilled in the art. For example, in the particular case where an implant is used to replace a nuclear pulposus, the nuclear material can be accessed by creating a hole in an annular fibrosis, followed by removal of the nuclear material by suction or other means. A delivery tube can then be used to deposit the implant at the site, followed by closure of the opening in the annular fibrosis and/or attachment of the nuclear implant. Some specific delivery techniques are discussed herein for use with embodiments of some implants described within this application.

Injectable SIS Formulations

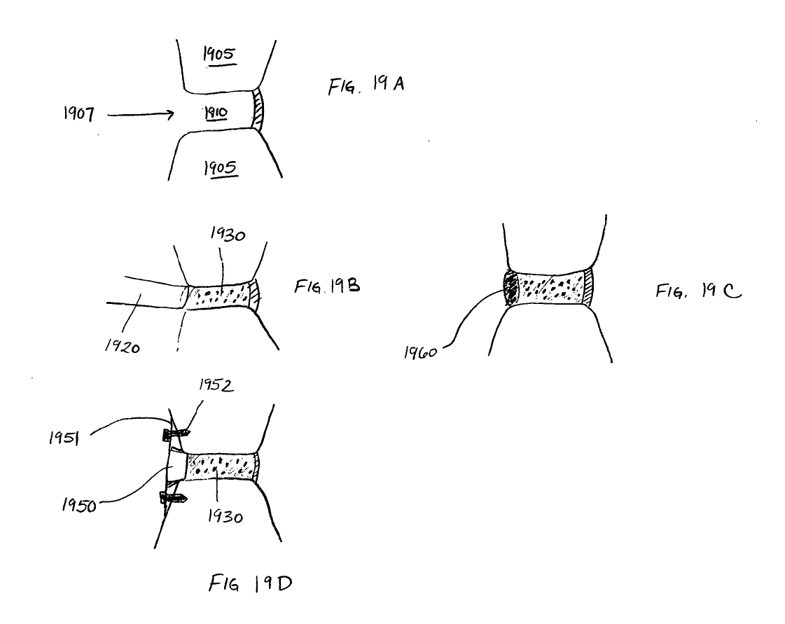

In another aspect, the invention is directed to small intestine submucosa (herein “SIS”) injectable formulations to provide an intervertebral implant. An exemplary use of SIS injectable formulations is illustrated in FIGS. 19A-19D. As shown in FIG. 19A, a damaged or diseased nuclear pulposus of an intervertebral disc is removed using known surgical techniques to form a void space 1910 between two vertebral bodies 1905 that is accessible by an opening 1907. Forming an opening can include the creation of a space in the annular fibrosis to provide access to the nucleus region. A delivery tube 1920 can be positioned at the opening 1907, as shown in FIG. 19B, to allow delivery of an injectable implant material 1930 that includes small intestine submucosa particulates. The injectable implant material 1930 can be provided in a quantity sufficient to exert a pressure in the void space that supports the intervertebral forces that act on a nominally functioning disc. After delivering the injectable material 1930, the opening 1907 can be closed using a plug-like structure 1950 held in place by securing devices 1952 that penetrate tab structure 1951 (shown in FIG. 19D) or some other mechanism suitable for covering the opening such as a closing material 1960 (shown in FIG. 19C). The closure 1950, 1960 hinders leakage of the injectable material 1930 from the void space 1910 and helps maintain the pressure in the void space to support intervertebral loading. An injectable implant material that includes SIS particulates can act to promote intervertebral tissue growth into the void region, while also promoting integration of the prosthesis with the patient's body.

The SIS particulates can be formed in a variety of dispositions not limited to particles, beads, chips, pellets, fibers, and/or strips. In some instances the particles used have relatively small sizes. For example, the median particle size may be in the range of about 1 micron to about 5 millimeters, or in the range of about 0.1 mm to about 3 mm, or in the range of about 1 mm to about 2 mm. In one example, the SIS particulates can be formed from collagen fibrils that are about 200 to about 300 microns long. Other particulates formed from resorbable materials (e.g., other extracellular materials besides SIS), non-resorbable materials, and/or biological materials can also be included in the formulation. Specific materials that can be used in an injectable formulation include all of the previously described resorbable materials, non-resorbable materials, cells (e.g., annular fibrosis cells, nuclear pulposus cells, and chondrogenic cells), bioactive factors, growth factors, and other materials used to construct biocompatible implant devices disclosed herein.

The injectable material can include a dispersal phase for dispersing particulates of the injectable formulation. Dispersal phases can be present in variety of dispositions including but not limited to liquids, gels, curables, slurries, putties, foams, cements, and other deformable phases. Gels, liquids, slurries of liquids and solids/gels, and other fluid-like dispersal phases can be especially suitable for filling the volume of an enclosure and exerting an outward pressure to resist intervertebral forces that act on the implant. Possible dispersants include organic and inorganic liquids (e.g., saline and/or hyaluronic acid), hydrogels (e.g., PVA, PVP, and/or PEG dispersed in liquids), polymers (e.g., polysilicones, polyurethanes, polyesters, polyacrylics, poly(propylene fumarates), dimethacrylated polyanhydrides, and poly(orthoesters)), and cement slurries (e.g., PMMA, TCP, hydroxyapatite, calcium sulfate, or solid particulates such as polymers, metallics, allo-, auto-, or xeno-bone grafts dispersed in a fluid phase). The amount and ratio of the various components can be controlled and adjusted based upon a variety of criteria that can include one or more of pre-surgical and/or surgical evaluations, disc pressure, disc height, and enclosure capacity. The volume fraction of solids in such injectable formulations are typically chosen to enhance flowing or deformation properties of the formulation, and can depend upon the size of the particulates as well. In some instances, the volume fraction of solids ranges from about 1% to about 50%, or from about 10% to about 40%, or about 20% to about 30%. Components such as alginate can be included in a filling material with SIS particulates to control the viscosity of the formulation. Also, the filling material can be formulated to promote fusion in the void space by the inclusion of osteoinductive materials and/or agents including those described herein.

With regard to any of these injectable SIS formulations, components and agents can be included to control transformation timing of an injectable formulation to a desired state. For example, when polymers are utilized, the injectable formulation can be presented as a polymer solution with crosslinking agent. After injection of the polymer solution into a disc region, crosslinking can be initiated utilizing heat, electrical current, ultrasound waves, radiation exposure, UV or visible light, or some other activation mechanism to create a gelled material. By way of one non-limiting example, the crosslinking agent can be encapsulated in a capsule that degrades after a certain amount of time, allowing the agent to immediately initiate crosslinking upon capsule degradation. In another example, osteoconductive agents or components can be incorporated into an injectable material, enabling the formation of a solid material after injection and curing. Other state transformations include increasing any one of the viscosity, density, elasticity, and/or modulus of the injectable material. One skilled in the art will appreciate that a variety of mechanisms are available to activate these changes of state of the injectable material after delivery to the implantation site.

Withdrawal of damaged or diseased intervertebral regions, and delivery of injectable SIS formulations can be achieved by any number of mechanisms including the use of specially modified syringe systems, nitinol delivery systems, and other existing systems currently used to perform angioplasty or to deliver catheters and stents. For example, a syringe system can be tailored to allow an injectable SIS formulation to be delivered to an intervertebral disc space following minimally invasive surgical procedures. The system can include a manifold in fluid communication with a first end of each of one or more delivery tubes. The manifold can include a valve to direct fluid movement toward one of the delivery tubes. Each of the delivery tubes can have an opposed end that is positioned proximate to an intervertebral region (e.g., a nuclear pulposus site) for delivering or withdrawing an injectable material. The manifold is in fluid communication with one or more fluid chambers that are each used to hold an injectable material, each fluid chamber having a corresponding piston effective to drive material into or out of the chamber.

In use, a portion or the entirety of a nuclear pulposus can be withdrawn from a disc space, via one of the delivery tubes, into one of the fluid chambers by suction created from a vacuum created by the chamber's associated piston movement. After nuclear pulposus withdrawal, the manifold valve can be redirected to allow fluid communication with another fluid chamber that contains the SIS injectable formulation. Subsequent corresponding piston movement then drives the SIS injectable formulation into the disc space. Alternatively, the SIS injectable formulation can be kept in two or more separate chambers for serial delivery. For example, one fluid chamber can deliver one portion of an SIS injectable formulation while another chamber subsequently delivers an osteoconductive carrier; thus allowing separation of components that can have a reactive timing feature that can be activated upon delivery at an implantation site. Other alternatives to the system include having an exchangeable fluid chamber assembly in connection with the manifold. This alternative can allow configurations with only one fluid chamber engaging the manifold. The fluid chamber is exchangeable between the steps of withdrawal or injection. In another alternative, a piston driver and controller can also be employed to allow controllable withdrawal and delivery of injectables from and to the implantation site. Use of a pressure and/or flow controller and associated sensors can be integrated with the system to help monitor intervertebral pressure during withdrawal and injection. One skilled in the art will appreciate that other concepts can also be applied to allow delivery of SIS injectables.

As alluded to by the representations in FIGS. 19C and 19D, a number of devices can be utilized to close or block an opening for delivering a SIS injectable formulation. In some embodiments, the devices include resorbable materials (such as SIS-based materials) that can facilitate the integration of the device with the patient's body. Accordingly, a plug-like structure, tab structures for holding securing devices, the securing devices themselves, or a closing material that cures quickly to form a seal can all include resorbable materials. It is understood, however, that the presence of a resorbable material is not a limitation to the types of closing or blocking devices that can be utilized.

Application of SIS injectable formulations have been described with respect to replacing a nuclear pulposus in the above-discussion. It is understood, however, that such formulations are not limited to this particular application. SIS injectable formulations can also be used to replace various spinal regions including a portion of, or the entirety of, a nuclear pulposus, an annular fibrosis, or a facet joint. Indeed, an SIS injectable formulation can suitably act as a replacement for an entire intervertebral disc. Injectable SIS formulations can also be utilized in conjunction with the SIS-based enclosure concepts presented herein, as well as other enclosures known to those skilled in the art, to provide other types of beneficial intervertebral augmentation devices and prostheses. Accordingly, an enclosure can be delivered to an implantation site, with a SIS injectable formulation being subsequently inserted into the enclosure for forming a desired implant. Alternatively, the enclosure can be delivered with the SIS injectable formulation already resident within the enclosure.

SIS-Based Collapsible Enclosure for Intervertebral Implants

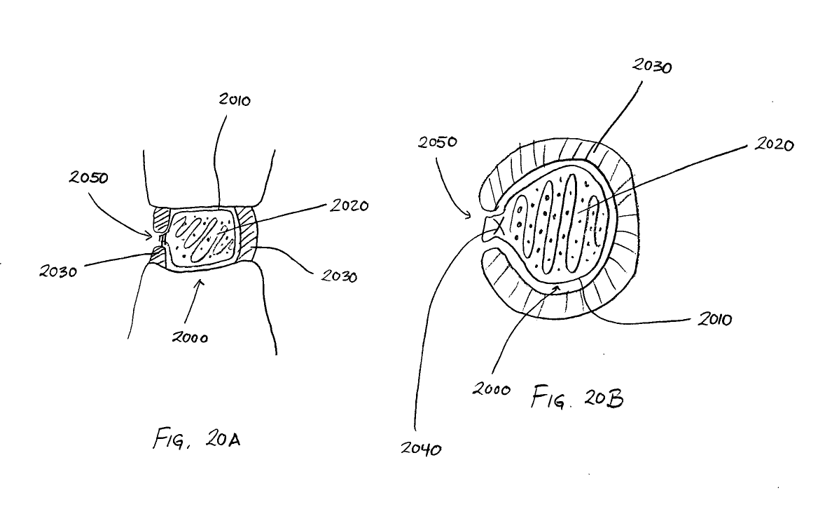

An additional aspect of the invention relates to intervertebral implants having a collapsible enclosure that holds a volume of filling material. FIGS. 20A and 20B respectively illustrate a transverse view and an axial view of one example of such an implant 2000 utilized as a nuclear pulposus prosthesis. The collapsible enclosure 2010 contains a filling material 2020 and is implanted within a intervertebral disc space that is surrounded by an annular fibrosis structure 2030. In general, the enclosure 2010 is filled with a sufficient quantity of filling material 2020 to expand the enclosure 2010 such that the implant 2000 is effective to sufficiently fill the disc space and to support intervertebral loading. The enclosure 2010 can include one port 2050 (though more than one can be present), which can be closed, using sutures 2040 or other suitable closure material including adhesives, after the enclosure 2010 is filled to a desired volume with filling material 2020.

In general, the collapsible enclosure construction can include one or more layers of small intestine submucosa (SIS). The SIS can act to promote intervertebral tissue growth and integration of the implant into the spinal region. Though the load bearing properties of the enclosure can be dictated by the pressure exerted by the filling material within the enclosure, load bearing properties can also be imparted by the enclosure construction. In one example, a collapsible enclosure can be constructed with one or more layers of load bearing material as previously described herein, such as a material having a higher compressive modulus than SIS when the device is implanted in the body. Such load bearing materials can also enhance the strength of the enclosure to hinder rupturing due to forces acting on the enclosure surfaces. In one example, the enclosure can be formed of a deformable laminate structure following any of the embodiments previously described. Accordingly, a collapsible enclosure can have additional layers of resorbable and/or non-resorbable materials, as well as layers having tissue ingrowth enhancement properties, adhesive properties, anti adhesion properties, etc. In another example, the enclosure strength of the enclosure can also be enhanced by utilizing a cross-hatch weave of layers in the enclosure's construction. The cross hatch weave can include weaved strips of SIS, or a mixture of strips of SIS and other materials (e.g., load bearing materials). SIS weaved strips can also be adapted such that the SIS strips do not have fibers that are all oriented in the same direction. The cross-hatch weave construction can also be combined with one or more laminate layers if desired.

Suitable filling materials can include one or more materials that can be used to expand and/or fill the volume of the collapsible enclosure. The filling material can be in a variety of states including solid, liquid, gas, slurry, gel, and combinations thereof. For example, filling materials can be pieces of tissue (e.g., folded SIS strips or sheets). Such tissue pieces can be mixed with a dispersal phase to form a volume-filing material that includes fluid. A volume filling material including liquid, gel, paste or other deformable, dispersal phase, with or without tissue, can be particularly advantageous by providing an even distribution of pressure on an enclosure to oppose intervertebral forces. SIS injectable formulations, as described herein, provide another useful category of filling materials. Indeed, any combination of the components described for the SIS injectable formulation can also be utilized herein to provide a suitable filling material.

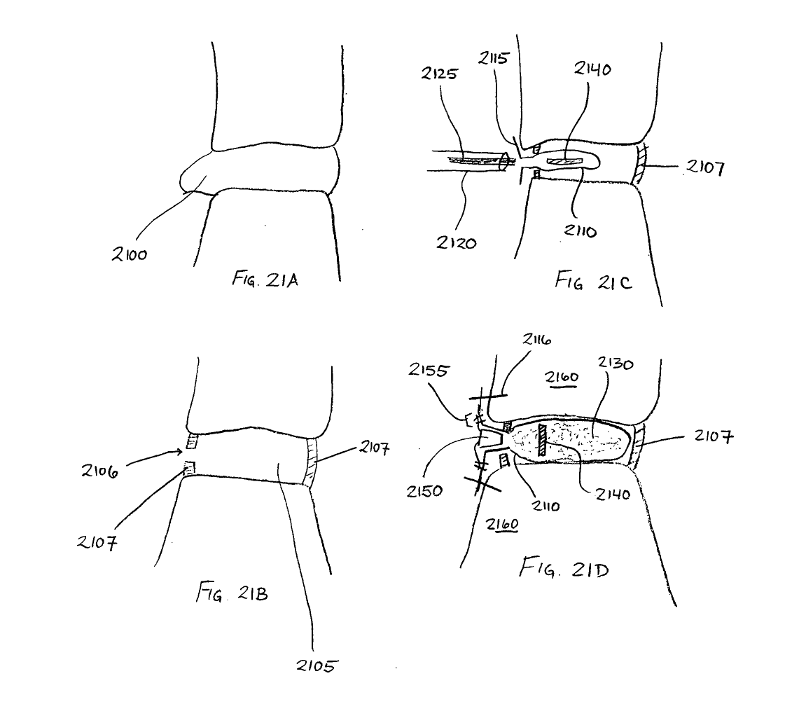

Though the port of the enclosure shown in FIGS. 20A and 20B can be closed with sutures, any other closure mechanism effective to hinder escape of filling material from the enclosure can be utilized so long as the mechanism is compatible for implantation into a patient's body. For example, a drawstring made from resorbable material (e.g., SIS fibers) and/or non-resorbable material can be coupled around each port of an enclosure to allow easy port closure by tensioning of the drawstring. Other closure mechanisms include utilizing fastening devices (e.g., pins, tacks, nails, and/or staples) to fasten the edges of the port to itself or to another part of the enclosure, to effect closure. A closing or blocking device can be utilized to block the port, one example being the plug structure 2150 being used to block the port of enclosure 2110 shown in FIG. 21D. One skilled in the art will also appreciate that one or more adhesives can be used to close the collapsible enclosure. Any effective combination of closure mechanisms can also be employed.

One example of the use of collapsible enclosures to provide a prosthesis for nuclear pulposus replacement is described with reference to FIGS. 21A-21D. Such prostheses can be advantageously delivered using minimally invasive surgical techniques. A herniated disc 2100, shown in FIG. 21A, is cleared to form an empty space 2105, shown in FIG. 21B, to be filled by a nuclear pulposus prosthesis. Removal of a diseased or damaged nuclear pulposus can be performed by forming a hole 2106 in an annular fibrosis 2107 as shown in FIG. 21B, followed by removal of the nuclear pulposus by suction or other techniques known to those skilled in the art. Alternatively, the annular fibrosis can also be removed when the prosthesis acts as an entire disc replacement device. As depicted in FIG. 21C, a collapsed enclosure 2110 can be advanced through a delivery tube 2120 using a pusher bar 2125. The tube 2120 is positioned to introduce the collapsed enclosure 2110 through the opening of the annular fibrosis 2107 and into the cleared space 2105. The pusher bar 2125 can be removed from the tube 2120 and filling material 2130 can then be introduced to expand and fill the collapsible enclosure 2110. The port of the collapsible enclosure 2110 can be blocked using a plug structure 2150 that is coupled to the enclosure 2110 by pins 2155. As shown in FIG. 21D, the enclosure 2110 can include one or more tab structures 2115 that are coupled to the port of the enclosure 2110. The tab structures 2115 can be used to attach the enclosure 2110 to bodily tissue. As shown in FIG. 21D, pins 2116 can be used to couple the enclosure 2110 to vertebral bone 2160, though other securing devices or adhesive can also be used. The enclosure could also be attached to soft tissue such as the annulus 2107. Tab structures can also be located elsewhere around the collapsible enclosure. Indeed, tab structures need not be utilized as the enclosure can be directly attached to tissue. Such attachment, though unnecessary, can help prevent unfavorable displacement of a prosthesis. In some instances, it is advantageous to attach the enclosure to tissue before inserting filling material into the enclosure to hinder prosthetic displacement during the filling process.

As shown in FIGS. 21C and 21D, an enclosure 2110 can include an expansion structure 2140 to help shape the enclosure. The expansion structure 2140 can either be preinserted into the enclosure before the enclosure is positioned in an intervertebral location, or it can be inserted into the enclosure after the enclosure is presented at the implantation site. As shown in FIG. 21C, the expansion structure 2140 can have an elongate shape (e.g., rod like) that allows easy delivery through a delivery tube 2120. When the collapsible enclosure is present at the implantation site, the expansion structure 2140 can be oriented to aid in expanding the enclosure, one example being the rotated expansion structure 2140 shown in FIG. 21D. The expansion structure can optionally be coupled to the enclosure to maintain a specific position relative to the enclosure. Furthermore, the expansion structure can be positioned relative to the vertebral bodies to provide a specific asymmetric loading profile for the prosthesis. FIG. 21D illustrates one example in which the expansion structure 2140 is positioned closer to the port end of the enclosure. This predetermined asymmetric loading profile of the prosthesis, i.e., the prosthesis can sustain more loading in one direction than another, can help redistribute intervertebral loading to correct abnormalities in vertebral orientation (e.g., lordosis or kyphosis). Expansion structures can be made from a variety of materials including resorbable materials which allow the structure to dissipate after a certain period of time. A person skilled in the art will appreciate that a number of different types of geometries and materials can be utilized that are consistent with the functionality described herein for an expansion structure. For example, two separate bodies can be utilized in conjunction to form the expansion structure.

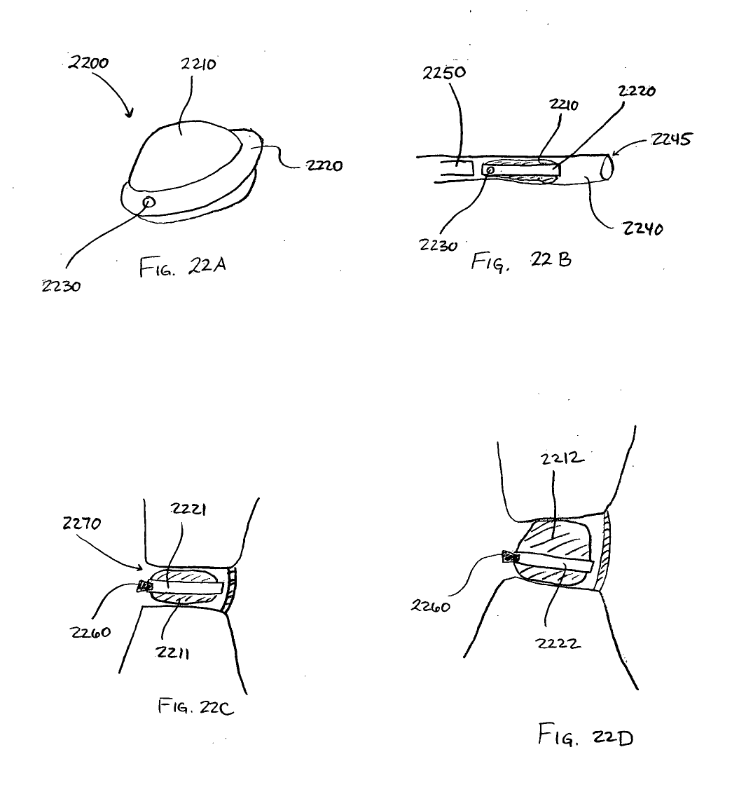

Another exemplary embodiment of an intervertebral implant is described with reference to FIGS. 22A-22D. As depicted in FIG. 22A, the implant 2200 can include a collapsible enclosure 2210 having at least one SIS layer (not shown), the enclosure 2210 having one or more ports. A collapsible support structure 2220 can be disposed around a peripheral portion of the collapsible enclosure 2210 to constrain the shape of the enclosure 2220 upon expansion. Such a support structure can also position the collapsible enclosure such that a desired orientation is achieved relative to the intervertebral implantation site upon enclosure expansion. For example, the support structure can hinder displacement of an expanding enclosure from a desired implantation site. A filling material (not shown) can be located within the collapsible enclosure 2210 effective to support loading on the implant 2210. In the embodiment shown in FIG. 22A, the collapsible support structure 2210 takes the form of a closed ring having an opening 2230 that is optionally aligned with a port (not shown) of the collapsible enclosure 2220.

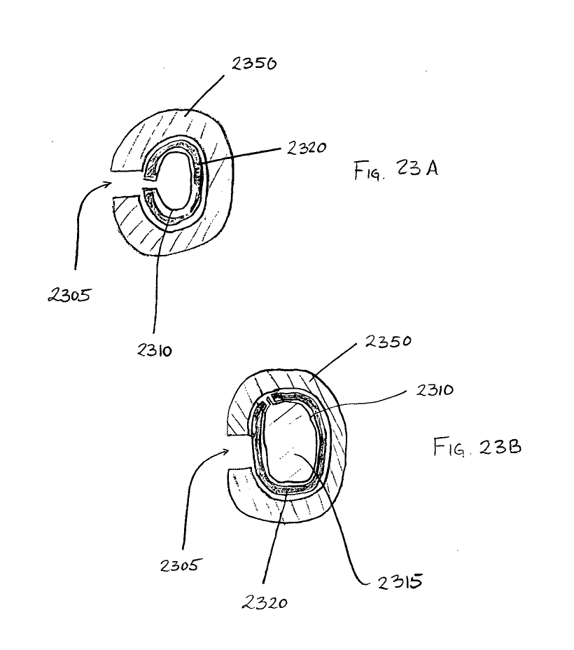

In use, the implant 2200 can be advanced within a delivery tube 2240 by means of a pusher 2250 to the distal end 2245 of the tube 2240 that is located adjacent to an implantation site 2270 as shown in FIGS. 22B and 22C. The collapsible support structure 2220 can be compressed by the walls of the delivery tube and constrained to fit within the inner diameter, the structure 2220 being capable of self-expansion upon emerging at the implantation site 2270 to a predetermined configuration. Upon delivery to an implantation site 2270, the support structure 2220 can optionally be attached to tissue or can maintain coupling with the delivery tube to secure its position at the implantation site 2270. The enclosure 2210 can be expanded by inserting a filling tube into the opening 2230 of the support structure to allow delivery of filling material into the collapsible enclosure 2210. The opening 2230 of the support structure 2220 is subsequently closed using a plug structure 2260 or any other type of closure mechanism or agent. Alternatively or in addition, a ring structure 2320 coupled to an enclosure 2310 can be aligned with a space 2305 formed in an annular fibrosis 2350 to allow filling of the enclosure 2310 as shown in FIG. 23A. Upon inserting filling material 2315 and closing the enclosure 2310, the ring structure 2320 can be rotated or repositioned, as depicted in FIG. 23B, such that the opening of the ring structure is not aligned with the space 2305 in the annular fibrosis 2350.

As previously discussed with reference to the use of expansion structures, collapsible support structures, along with their associated collapsible enclosures, can also be configured and oriented to provide a prosthesis that has a predetermined asymmetric loading profile. That is, the prosthesis can have preferred directions for sustaining loading that can be beneficial for addressing intervertebral conditions such as lordosis and kyphosis. As shown in FIG. 22C, the support structure 2221 and enclosure 2211 are adapted to sustain the intervertebral loading in a symmetric manner. The support structure 2222 and enclosure 2212 shown in FIG. 22D, however, is biased to provide more support to the region where the vertebral bodies are in closer proximity to help cure the misalignment of the vertebral bodies. A person skilled in the art will appreciate that variations in the sizes, shapes, and relative positions of the support structure and collapsible enclosure, as well as the positioning of the prosthesis at the implantation site, can all be adjusted accordingly to provide a predetermined asymmetric loading profile for the implant.

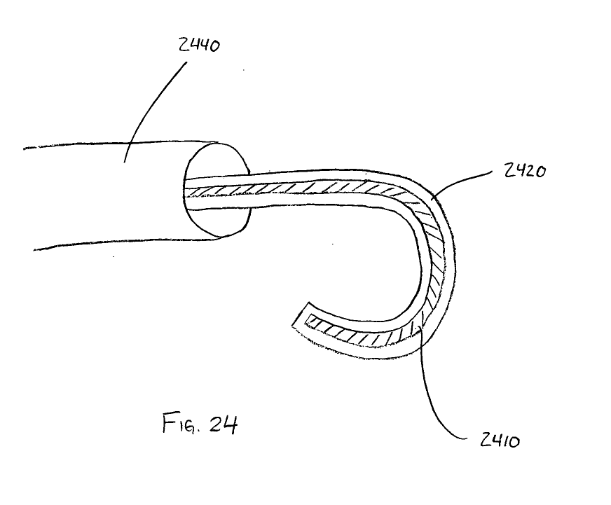

Collapsible support structures effective to constrain a collapsible enclosure to an expanded shape can be embodied by a variety of techniques, including and beyond the specific structures depicted in FIGS. 22A-22D. For example, as depicted in FIG. 24, an open ring support structure 2420, attached to a collapsible enclosure 2410, can be deployed through a delivery tube 2240 to an implantation site. The open ring structure can be formed with an elastic material such that the support structure 2420 and collapsible enclosure 2410 can be extended in the longitudinal direction of the axis of the delivery tube 2440 while being transported therethrough. Elastic material, when utilized in a collapsible support structure, can generally made the structure pliable and deformable. Upon emerging from the tube at the implantation site, the support structure 2420 can curl into an open ring configuration since the elastic material tends to bias the support structure 2420 into a predetermined configuration. Beyond the use of an elastic material, such as metal alloys or other materials with superelasticity which can be utilized in both open and closed ring support structures, other materials such as shape memory materials can also be used to create collapsible structures that are reversibly deformable. One type of a shape memory material includes nickel-titanium alloys that are thermally activated. Other shape memory materials can be activated by pressure contact, heat, electrical current, ultrasound waves, radiation exposure, and/or UV or visible light. Other variations include devices where the support structure and collapsible enclosure are not physically attached. In such an instance, the support structure can be delivered to the implantation site with or without the enclosure. As the collapsible enclosure is expanded with filling material, the support structure acts to maintain a particular shape of the enclosure and to position the enclosure in a particular orientation relative to the implantation site. After expanding the enclosure, the support structure can be removed or left behind as part of the collapsible enclosure for continued structural support. When a support structure is to be held in a particular position at an implantation site, a variety of attachment devices (e.g., screws, pins, sutures, tacks, adhesives, staples, etc.), made of nonresorbable or resorbable materials, can be utilized to attach the support structure to tissue. Attachment mechanisms include those described herein for use with other exemplary prostheses such as tab structures.

Collapsible support structures can be constructed of a variety of materials compatible with the functioning of the structure. For example, support structures can be constructed of the same materials from which an enclosure can be constructed (e.g., resorbable materials, non-resorbable materials, and combinations thereof as described herein). In particular, resorbable materials such as ECM and SIS layers can allow the support structure to be integrated with a patient's body with time. Resorbable or non-resorbable materials in a collapsible support structure can sustain substantial loading with predetermined degradation rates. Non-limiting examples of such structures include a coated spring-like metal, a polymeric spring, and a polymeric or metallic expandable cage. Collapsible support structures can also include the use of polymers such as PEEK.

Those skilled in the art will appreciate that the collapsible enclosures and filling materials that can be utilized with a collapsible support structure include all those described in previous embodiments herein, along with their associated auxiliary features. Accordingly, for example, collapsible enclosures with a layer of SIS and a load bearing material can be implemented with a support structure. Enclosures that include layers or coatings of materials such as tissue ingrowth promoters are also within the scope of the present invention. As well, securing devices, tab structures, closing/blocking devices, and other features, as revealed herein or known to those skilled in the art, can be combined with an enclosure and a support structure.

SIS-Based Self-Shaping Intervertebral Implants

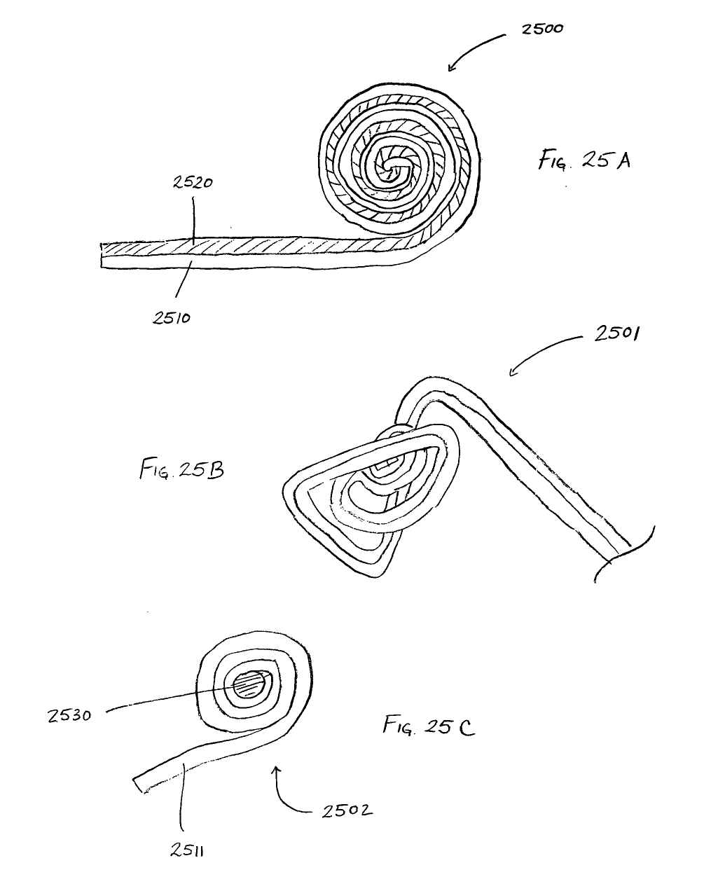

Further embodiments are directed toward devices and methods regarding intervertebral implants, or portions thereof, having a hybrid implant structure. One exemplary embodiment is described with reference to FIG. 25A in which an intervertebral implant 2500 includes the use of a hybrid structure having a tissue regeneration structure and a shaping structure coupled together. The tissue regeneration structure can include at least one layer of SIS, and aids the promotion of tissue ingrowth. Tissue regeneration structures can also include additional materials, as described elsewhere herein. The shaping structure can include a self-shaping material, such as an elastic material, and acts to bias the implant toward a predetermined, at-rest configuration. By way of example, for the implant 2500 shown in FIG. 25A, the tissue regeneration structure is embodied as a SIS layer 2520 that comprises one or more sheets of SIS, and the shaping structure is embodied as an elastic layer 2510 that is coupled to the SIS layer. Thus, the hybrid implant structure resembles a self-coiling laminate, and the elastic layer 2510 biases the hybrid implant structure toward the coiled configuration. The hybrid implant structure, however, can be unraveled into an elongate shape upon application of a stimulus along the elongate direction of the laminate. In use, the implant 2500 can be unraveled and kept in an elongated configuration while being transferred in a delivery tube, or other conduit, to an implantation site. Upon emerging from the delivery tube, the self-shaping nature of the shaping structure tends to coil the implant into a coiled configuration. Accordingly, such an implant can provide a convenient manner for forming a desired implant shape while delivering the implant using minimally invasive techniques.

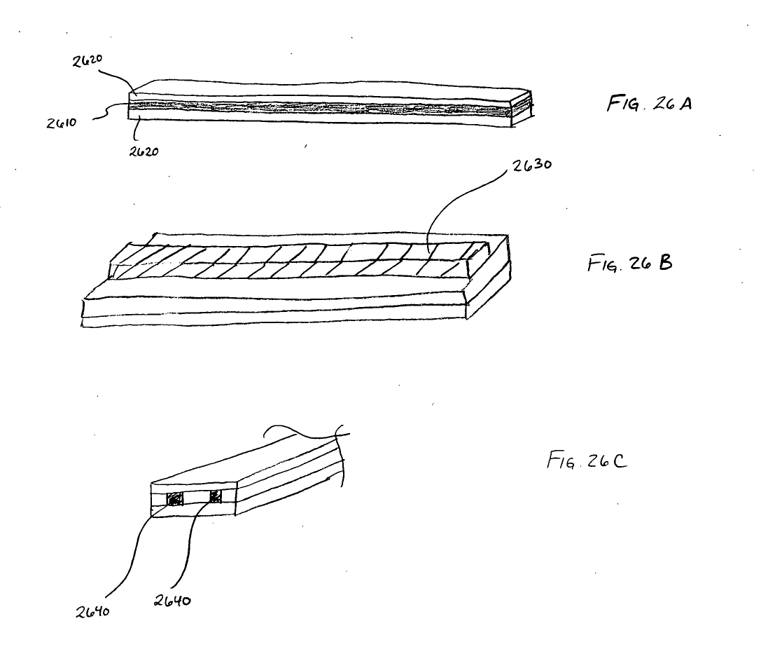

The shaping structures used to form hybrid implant 2500 can include any materials that can bias an implant toward a predetermined, at-rest, configuration. Though elastic materials, such as materials which have superelastic properties, can be used effectively, other materials such as shape memory materials can also be utilized. Such shape memory materials can include nickel titanium alloys which retain a specific shape when exposed to a particular thermal environment, as well as materials that respond to other stimuli such as pressure contact, electrical current, ultrasound waves, radiation exposure, and UV and/or visible light. Combinations of self-shaping materials can also be formed into shaping structures. For example, two different types of elastic materials with different elastic modulus values can be implemented into different sections of an implant to cause tighter bending in some regions relative to others. Shaping structures can be embodied as one or more continuous layers, as exemplified by the elastic layer 2610 sandwiched between two SIS layers 2620 shown in FIG. 26A. Shaping structures can also be embodied as one or more strips, as depicted by the strips 2630, 2640 shown in FIGS. 26B and 26C. The predetermined at-rest configuration to which the implant is biased by the shaping structure need not be a coiled configuration. Indeed, the at-rest configuration can be a folded configuration as depicted by the implant in FIG. 9A, a more randomized volumetric configuration 2501 as depicted in FIG. 25B, or any other type of raveled configuration that is predetermined for use at an implantation site.

It is understood that a variety of features associated with other implant embodiments discussed herein can be utilized with an implant having the hybrid implant structure previously discussed. For example, a load bearing structure can be coupled to the hybrid structure such as to be effective to support loading on the implant. The load bearing structure can include any of the properties and configurations discussed previously herein. Accordingly, an implant having the load bearing properties associated with a load bearing structure can also be afforded the properties associated with having a shaping structure. In another example, a hybrid implant can be combined with one or more additional layers and/or coatings as described herein for use with laminate structures and other implants. Accordingly, some of the coatings or layers can include adhesives, anti-adhesives, biofactors, tissue ingrowth enhancing components, cells, osteoinductive materials, or other components. Furthermore, block structures, as described herein, can also be included with hybrid implant structure. In one example, illustrated in FIG. 25C, a block structure is embodied as a core 2530 that is coupled to one end of the hybrid implant structure 2511, and the hybrid implant structure 2511 is adapted to ravel around the core 2350 as shown in FIG. 25C. Other features, such as the use of tab structures and/or securing devices, can also be implemented with a hybrid implant structure.

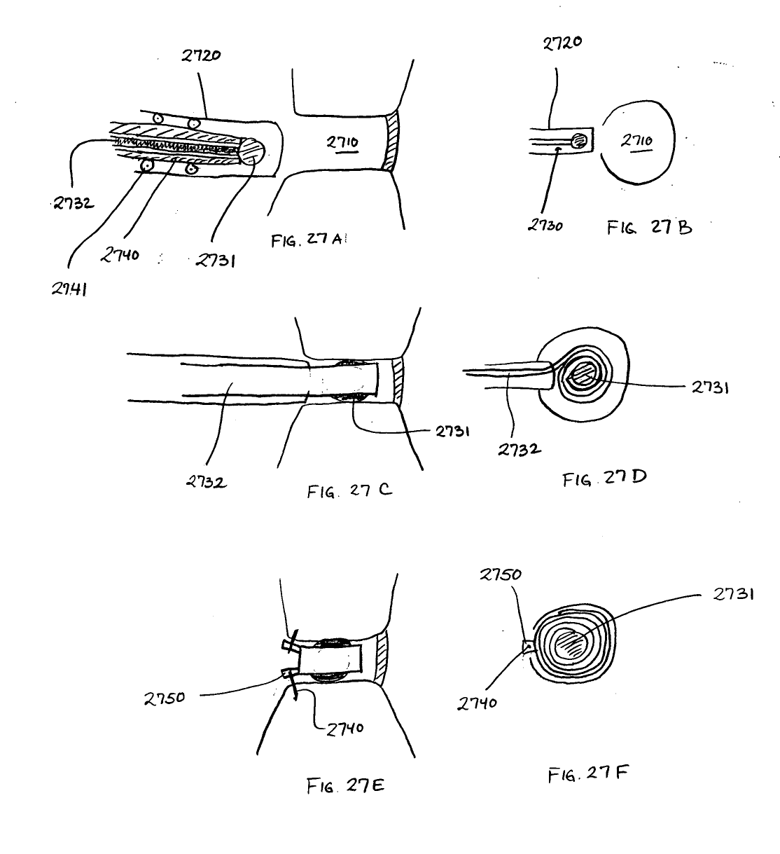

An exemplary method of delivering an intervertebral implant with a hybrid implant structure is described with reference to FIGS. 27A-27F. FIGS. 27A, 27C, and 27E present transverse views of various stages of implant insertion, while FIGS. 27B, 27D, and 27F present the corresponding axial views, respectively. According to one exemplary technique, an intervertebral region can be cleared to provide a void space 2710 for implant placement. A hybrid implant 2730 can be deformed from its raveled, at-rest configuration to an elongate shape that can be inserted within a delivery tube 2720 as depicted in FIGS. 27A and 27B. Delivery of the elongated hybrid implant can be achieved using a hollow pusher, such as the annular pusher 2740 shown in FIG. 27A. The uncoiled laminate 2732 can be threaded within the annular space of the pusher 2740, with the core 2731 of the implant 2730 positioned at the distal end of the pusher 2740. The ensemble of the pusher 2740 and the unwound hybrid implant 2730 can then be advanced through the delivery tube 2720 to the implantation site 2710 with the core 2731 leading the entire assembly. Optionally, one or more rollers 2741 can be utilized to guide and facilitate movement of the pusher 2740 through the delivery tube 2720. Upon positioning the core 2731 at the implantation site 2710, withdrawal of the pusher 2740 can initiate laminate 2732 winding around the core 2731 as shown in FIGS. 27C and 27D. After the laminate 2732 is completely wound, the hybrid implant 2730 can be optionally secured at the implantation site by securing devices 2740 into tab structures 2750, which are attached to the hybrid implant 2730, as illustrated in FIGS. 27E and 27F. Modification and augmentation of the exemplary method can be achieved without straying from the scope of the present invention. For example, other types of pushers or delivery mechanisms and/or devices can be employed to deliver an unraveled hybrid implant to an implantation site.

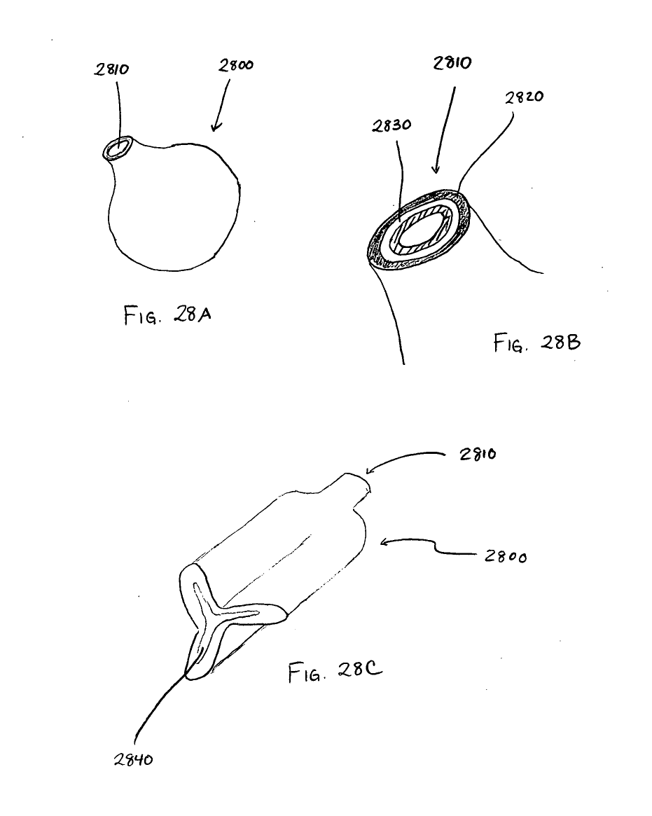

Another exemplary embodiment is drawn to an intervertebral implant that includes a tissue regeneration structure and a shaping structure, which are coupled and configured to form a reversibly deformable enclosure. The reversibly deformable nature of the enclosure can bias the implant toward a predetermined configuration, such as an expanded configuration as illustrated by the enclosure 2800 depicted in FIG. 28A. With reference to the close up of view of the opening 2810 of the enclosure 2800 shown in FIG. 28B, the enclosure 2800 includes a tissue regeneration structure 2820 for promoting tissue ingrowth embodied as a layer conforming to the shape of the enclosure 2800. The tissue regeneration structure can include at least one layer of SIS that can be configured as a SIS layer coupled to the surface of a shaping structure 2830. The shaping structure 2830, which can be used to bias the implant toward the expanded configuration, can include a self-shaping material, such as an elastic material configured as a layer. In general, shaping structures can utilize any of the materials and geometries discussed with respect to other embodiments disclosed herein. For example, the shaping structure can be a plurality of strip like structures that are biased toward a particular configuration. As well, the enclosure can include any other additional features discussed in other embodiments revealed herein with respect to enclosures (e.g., additional openings, tab structures disposed at an opening to the enclosure, securing devices optionally constructed with SIS or other resorbable materials, injectable SIS or other filling materials disposed within the enclosure, etc.).

Implant enclosures including a shaping structure (herein also called “hybrid enclosures”) can be deformed into a collapsed shape that can be disposed within a hollow delivery device. One example of a collapsed shape is depicted in FIG. 28C, showing a cutaway view of the enclosure 2800 opposite the opening 2810. The enclosure 2800 can be collapsed and folded, reducing its internal volume 2840 and allowing the enclosure 2800 to fit within a hollow delivery device.

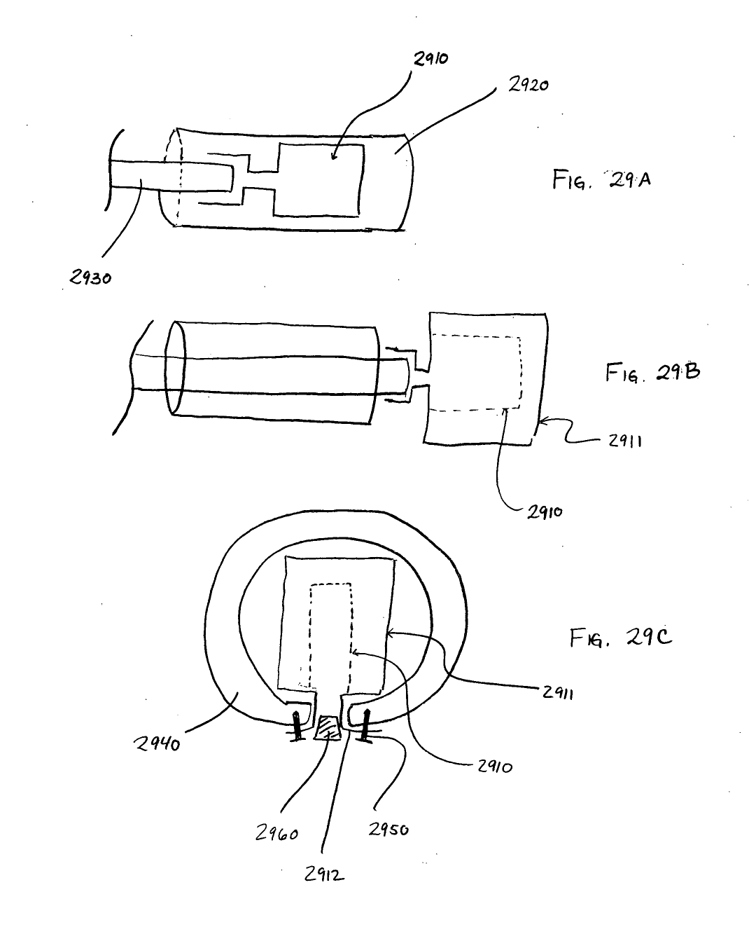

FIGS. 29A-29C provide an exemplary depiction of the deployment of a hybrid enclosure. According to this exemplary technique, a hybrid enclosure can be deformed into a collapsed shape 2910 to fit within a delivery tube 2920 as depicted in FIG. 29A. The hybrid enclosure can be advanced through the delivery tube 2920 with the use of a pusher 2930. With reference to FIG. 29B, upon emerging from the delivery tube at an implantation site, the hybrid enclosure can self-expand from the collapsed shape 2910 toward an expanded, at-rest configuration 2911. An axial view of the deployed hybrid enclosure is presented in FIG. 29C, with the enclosure attached to an annular fibrosis 2940 by pins 2950 and tab structures 2912. The enclosure can be subsequently loaded with a filling material, and closed with a plug structure 2960. Of course, other variations regarding the use of enclosures as discussed elsewhere herein can be employed with the use of hybrid enclosures. The specific descriptions and depictions of FIGS. 29A-29C are not meant to limit the scope of use of hybrid enclosures.

Persons skilled in the art will understand that the devices and methods specifically described herein and illustrated in the accompanying drawings are non-limiting exemplary embodiments. The features illustrated or described in connection with one exemplary embodiment may be combined with the features of other embodiments. Such modifications and variations are intended to be included within the scope of the present invention. As well, one skilled in the art will appreciate further features and advantages of the invention based on the above-described embodiments. Accordingly, the invention is not to be limited by what has been particularly shown and described, except as indicated by the appended claims.

What is claimed is:

1. An intervertebral implant comprising:
a hybrid implant structure including
(i) a tissue regeneration structure for promoting tissue ingrowth comprising at least one small intestine submucosa layer; and
(ii) a shaping structure for biasing the implant toward a predetermined, at-rest configuration comprising an elastic material,
the tissue regeneration structure and the shaping structure coupled together to form a reversibly extendable structure having an elongate shape upon the application of a force along an elongate direction of the elongate shape.

2. The intervertebral implant of claim 1, further comprising:
a load bearing structure coupled to the hybrid implant structure and effective to support a load on the intervertebral implant, the load bearing structure comprising a load bearing material having a higher compressive modulus than small intestine submucosa.

3. The intervertebral implant of claim 1, wherein the elastic material is configured as at least one continuous layer.

4. The intervertebral implant of claim 1, wherein the elastic material is configured as at least one strip.

5. The intervertebral implant of claim 1, wherein the predetermined, at-rest configuration is a raveled configuration adapted to be implanted at least within a portion of an intervertebral space.

6. The intervertebral implant of claim 5, wherein the predetermined, at-rest configuration is a coiled configuration.

7. The intervertebral implant of claim 5, further comprising:
a core coupled to one end of the reversibly extendable structure, wherein the reversibly extendable structure is adapted to ravel around the core.

8. The intervertebral implant of claim 1, wherein the reversibly extendable structure includes at least one tab structure effective to receive a securing device for attaching the intervertebral implant to tissue.

9. An intervertebral implant comprising:
a tissue regeneration structure for promoting tissue ingrowth comprising at least one small intestine submucosa layer, the tissue regeneration structure having a surface; and
a shaping structure for biasing the implant toward a predetermined configuration comprising an elastic material, the shaping structure having a surface,
the surfaces of the tissue regeneration structure and shaping structure coupled and adapted to form a reversibly deformable enclosure having at least one opening.

10. The intervertebral implant of claim 9, wherein the predetermined configuration disposes the reversibly deformable enclosure in an expanded configuration.

11. The intervertebral implant of claim 9, further comprising:
a filling material located within the reversibly deformable enclosure effective to support loading on the implant.

12. The intervertebral implant of claim 9, wherein the reversibly deformable enclosure includes at least one tab structure effective to receive a securing device for attaching the intervertebral implant to tissue.

13. The intervertebral implant of claim 12, wherein the at least one tab structure is disposed toward the at least one opening of the reversibly deformable enclosure.

14. The intervertebral implant of claim 9, further comprising a plug structure effective for at least partially blocking the at least one opening of the reversibly deformable enclosure.

15. A method of delivering an intervertebral prosthesis to an implantation site, comprising:
providing a hybrid implant biased toward a raveled, at-rest configuration, and comprising small intestine submucosa and an elastic material;
deforming the hybrid implant to an elongate shape effective to be disposed in an elongate, hollow delivery device;
delivering the hybrid implant to a desired implantation site through the delivery device; and
depositing at least a portion of the hybrid implant at the implantation site such that the hybrid implant self-ravels toward the raveled, at-rest, configuration upon removal from the delivery device.

16. The method of claim 15, wherein the raveled, at-rest configuration is a coiled configuration.

17. The method of claim 15, wherein delivering the hybrid implant includes delivering the hybrid implant through the delivery device using at least one roller.

18. A method of delivering an intervertebral prosthesis to an implantation site, comprising:
providing a hybrid enclosure having at least one opening and biased toward an expanded, at-rest configuration, the hybrid enclosure comprising small intestine submucosa and an elastic material;
deforming the hybrid enclosure to a collapsed shape effective to be disposed in an elongate, hollow delivery device;
delivering the hybrid enclosure to a desired implantation site through the deliver device; and
depositing the hybrid enclosure at the implantation site such that the hybrid enclosure self-expands toward the expanded, at-rest, configuration upon removal from the delivery device.

19. The method of claim 18, further comprising:
filling the hybrid enclosure with a filling material; and
closing the opening of the hybrid enclosure.

20. The method of claim 18, further comprising:
attaching the hybrid enclosure to a portion of a spine at the opening of the hybrid enclosure.
